(19) United States
(12) Patent Application Publication
(10) Pub. No.: US 2024/0358867 A1
(43) Pub. Date: Oct. 31, 2024

(54) REAGENTS FOR SITE-SPECIFIC LABELING OF PROTEINS WITH RADIOHALOGENS, AND METHODS OF MAKING AND USING THE SAME

(71) Applicants: DUKE UNIVERSITY, DURHAM, NC (US); THE RESEARCH FOUNDATION OF THE CITY UNIVERSITY OF NEW YORK, NEW YORK, NY (US)

(72) Inventors: MICHAEL ZALUTSKY, DURHAM, NC (US); YUTIAN FENG, DURHAM, NC (US); GANESAN VAIDYANATHAN, DURHAM, NC (US); BRIAN ZEGLIS, NEW YORK, NY (US); SAMANTHA SARRETT, NEW YORK, NY (US)

(73) Assignees: DUKE UNIVERSITY, DURHAM, NC (US); THE RESEARCH FOUNDATION OF THE CITY UNIVERSITY OF NEW YORK, NEW YORK, NY (US)

(21) Appl. No.: 18/687,784

(22) PCT Filed: Aug. 31, 2022
(86) PCT No.: PCT/US2022/042257

§ 371 Date: Feb. 28, 2024

Related U.S. Application Data
(60) Provisional application No. 63/279,355, filed on Nov. 15, 2021.
(60) Provisional application No. 63/239,463, filed on Sep. 1, 2021.

(51) Int. Cl. A61K 51/10; C07B 59/00; C07K 1/107

(52) U.S. Cl. CPC A61K 51/1096; A61K 51/1045; C07B 59/00; C07K 1/1077; A61K 2121/00; A61K 2123/00; C07B 2200/05

(57) ABSTRACT

The present disclosure provides, in part, methods for site-specific labeling of proteins/peptides with radiohalogens and compositions resulting from said methods.

### CROSS-REFERENCE TO RELATED APPLICATIONS

This application claims the benefit priority to U.S. Provisional Application No. 63/239,463 filed Sep. 1, 2021, and U.S. Provisional Application No. 63/279,355 filed Nov. 15, 2021. The entire contents of the foregoing applications are hereby incorporated herein in their entirety.

### FIELD OF THE INVENTION

The subject matter disclosed herein generally relates to site-selective radiolabeled biological molecules that are stable in vivo.

### BACKGROUND OF THE INVENTION

The ability of many biomolecules to selectively and specifically target cellular biomarkers has long been harnessed for both molecular imaging (e.g., positron emission tomography, “PET”) and targeted radiotherapy in which a biomolecule is labeled with a radioactive atom for selective delivery to cancer cells with the goal of killing them. This exploitation uses the ligation of a radioactive payload to the biomolecule. Some of the best radionuclides for this purpose are radiohalogens. For example, ¹⁸F is used for PET imaging, ¹³¹I is used for beta-particle therapy, and ²¹¹At is used for alpha-particle therapy.

In the case of peptides and proteins, radiolabeled molecules are typically prepared by ligation of reactive, bifunctional probes to amino acids within the biomolecule, most often lysines. While controlling the site of this conjugation is fairly easy with small peptides which rarely possess more than one or two copies of each amino acid this becomes a much bigger problem with larger biomolecules. For example, most antibodies contain dozens of lysines distributed throughout their macromolecule structure. The indiscriminate attachment of radioactive molecules or a precursor molecule that will be labeled in a subsequent step to these lysines can lead to the formation of thousands of different protein conjugates that differ in the location of the lysine that is modified and/or the number of different lysines in an individual protein that are modified. Not surprisingly, this so-called “random” approach to bioconjugation has low reproducibility and produces a highly heterogeneous population of conjugates. In addition, this strategy can result in payloads being inadvertently grafted to bioactive sites in the biomolecule, rendering a portion of the conjugates inoperative.

To circumvent these issues, researchers have turned their attention to strategies allowing for better control over the site of the ligation reaction, known as “site-specific” bioconjugations. Conjugations to cysteine—a thiol-bearing amino acid present in small numbers in proteins—with maleimide-bearing probes have become a staple of this field and have been used extensively over the last three decades. The prevalence of this strategy is rooted mainly in its simplicity and efficiency. Many proteins contain disulfide bridges that can be easily reduced to form the reactive thiols, or a free cysteine can be added to the protein using standard recombinant technologies. The Michael addition between the maleimide and the sulfhydryl group then can be performed at physiological pH and room temperature, reliably leading to the formation of a succinimidyl thioether linkage within an hour (see FIG. 1).

While the ligation between thiols and maleimides represents an undeniable improvement over random conjugation methods, the reaction suffers from drawbacks as well. Indeed, maleimide-based conjugates display limited stability in physiological media because the conjugate can undergo a retro-Michael reaction that leads to the release of the payload or its transfer to other endogenous molecules containing free thiols (most often serum albumin, cysteine, and glutathione) by transthiolation. In the context of nuclear imaging and radiotherapy, this retro-Michael reaction can lead to the release of the radioactive payload and in vivo radiolabeling of endogeneous biomolecules through thiol exchange reactions (see FIG. 1). This “leakage” causes higher uptake in non-target tissues and concomitant lower uptake in target tissues, ultimately resulting in higher radiation doses to healthy tissues, reduced imaging contrast, and lower target-to-normal tissue radiation dose ratios, and as a result, diminished therapeutic effectiveness.

It would therefore be desirable to create a method of radiolabeling proteins, at a specific site on the protein, using a linking strategy that is both selective and stable under the physiological conditions experienced at the desired target site.

The discussion above is merely provided for general background information and is not intended to be used as an aid in determining the scope of the claimed subject matter.

### SUMMARY OF THE INVENTION

The present disclosure provides novel precursors for radiohalogenation, processes for preparing the radiohalogenated prosthetic agents, and processes for using the radiohalogenated prosthetic agents to prepare a radiolabeled protein or peptide.

In an embodiment, a radiohalogenated prosthetic agent or a radiohalogenation support precursor has the structure (I):

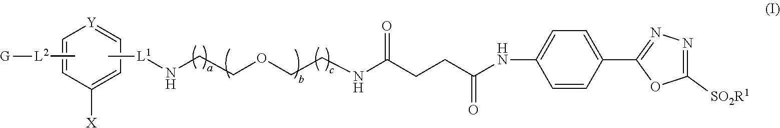

where:
a is 1-6; b is 1-6; c is 1-6;
L¹ is —C(O)— or

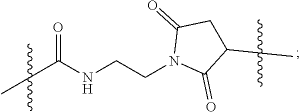 [SMILES: *C(=O)NCCN1C(=O)CC(*)C1=O]

L² is a bond or —(CH₂)ₙ—, where n is 1-6;
G is guanidine, a guanidine having one or more carbamate protecting groups, PO₃H, SO₃H, PO₂—
OtBu, SO₂—OtBu, arginine, phosphono-phenylalanine, sulfo-phenylalanine, glutamate, aspartate, lysine, a hydrophilic carbohydrate moiety, or a polyethylene glycol (PEG) chain;
Y is CH or N;
X is SnR²³, B(OH)₂, Bpin, or a radiohalogen;
R¹ is C₁₋₆ alkyl; and
each R² is independently C₁₋₆ alkyl.

In an embodiment, a radiohalogenated prosthetic agent or a radiohalogenation support precursor has the structure (II):

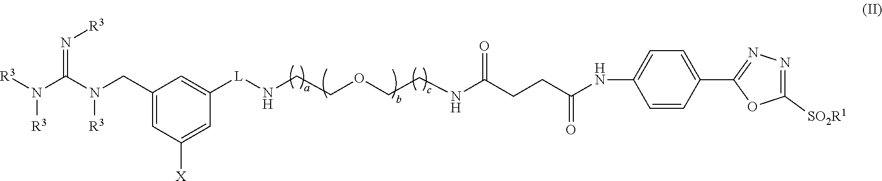

where:
a is 1-6; b is 1-6; c is 1-6;
L is —C(O)— or

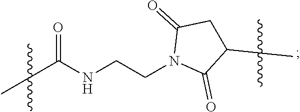 [SMILES: *C(=O)NCCN1C(=O)CC(*)C1=O]

X is SnR²³, B(OH)₂, Bpin, or a radiohalogen;
R¹ is C₁₋₆ alkyl;
each R² is independently C₁₋₆ alkyl; and
each R³ is independently a carbamate protecting group or H.

In an embodiment, a radiohalogenated prosthetic agent or a radiohalogenation support precursor has the structure (III):

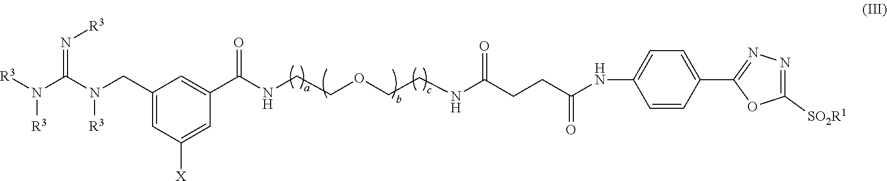

In an embodiment, a radiohalogenated prosthetic agent or a radiohalogenation support precursor has the structure (IV):

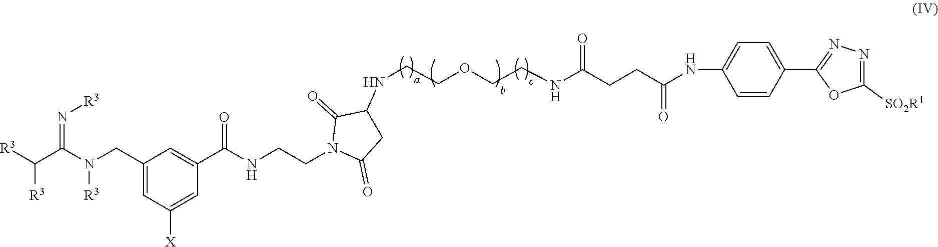

In an embodiment of the present disclosure, R¹ in any of the compounds (I)—(IV) is methyl, ethyl, or propyl. In an embodiment of the present disclosure, a is 1 or 2, b is 2 or 3, and c is 1 or 2, in any of the compounds (I)—(IV). In an embodiment of the present disclosure, X in any of the radiohalogenation support precursors of compounds (I)—(IV) is SnR²₃, B(OH)₂, or Bpin, where R² is methyl, ethyl, or n-butyl. In an embodiment of the present disclosure, X in any of the radiohalogenated prosthetic agents of compounds (I)—(IV) is selected from the group consisting of ¹⁸F, ¹²²I, ¹²³I, ¹²⁴I, ¹²⁵I, ¹³¹I, ⁷⁵Br, ⁷⁶Br, ⁷⁷Br, ^{80m}Br, and ²¹¹At.

In an embodiment, radiolabeled protein or peptide includes one or more radiohalogenated prosthetic agents coupled to a protein or peptide through a thioether bond, wherein the one or more radiohalogenated prosthetic agent-protein/peptide conjugates have structure (V), or the one or more radiohalogenated prosthetic agent-protein/peptide conjugates are a pharmaceutically acceptable salt of structure (V):

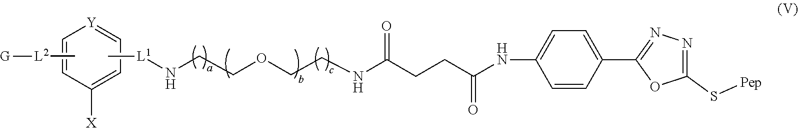

where:
a is 1-6; b is 1-6; c is 1-6;
L¹ is —C(O)— or

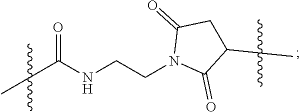 [SMILES: *C(=O)NCCN1C(=O)CC(*)C1=O]

L² is a bond or —(CH₂)ₙ—, where n is 1-6;
G is guanidine, a guanidine having one or more carbamate protecting groups, PO₃H, SO₃H, PO₂—OtBu, SO₂—OtBu, arginine, phosphono-phenylalanine, sulfo-phenylalanine, glutamate, aspartate, lysine, a hydrophilic carbohydrate moiety, or a polyethylene glycol (PEG) chain;
Y is CH or N;
X is a radiohalogen;
R¹ is C₁₋₆ alkyl; and
Pep is a protein or a peptide.

In an embodiment, a radiolabeled protein/peptide has the structure (VI):

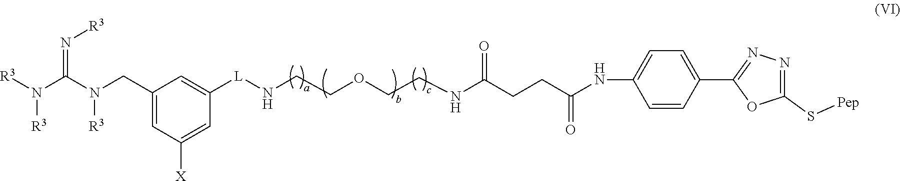

where:
a is 1-6; b is 1-6; c is 1-6;
L is —C(O)— or

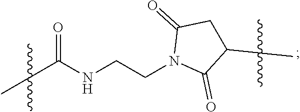 [SMILES: *C(=O)NCCN1C(=O)CC(*)C1=O]

X is a radiohalogen;
R¹ is C₁₋₆ alkyl;
each R³ is independently a carbamate protecting group or H; and
Pep is a protein or peptide.

In an embodiment, a radiolabeled protein/peptide has the structure (VII):

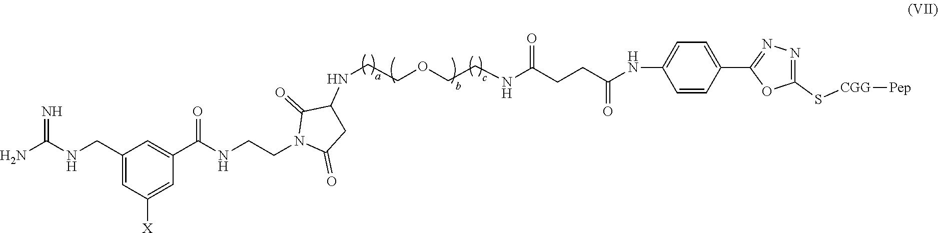

where Pep is a protein/peptide.

In an embodiment, a radiolabeled protein/peptide has the structure (VIII):

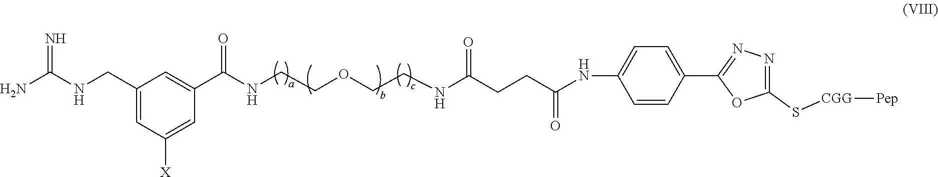

where Pep is a protein/peptide.

In an embodiment of the present disclosure, X in any of the compounds (V)—(VIII) is selected from the group consisting of ¹⁸F, ¹²²I, ¹²³I, ¹²⁴I, ¹²⁵I, ¹³¹I, ⁷⁵Br, ⁷⁶Br, ⁷⁷Br, ^{80m}Br, and ²¹¹At. In an embodiment of the present disclosure, the protein/peptide in any of the compounds (V)—(VIII) comprises at least one cysteine residue and wherein one or more of the radiolabels are coupled to the protein/peptide through the cysteine residue. In an embodiment to the present disclosure, the protein/peptide comprises a C-terminal glycine-cysteine tail and wherein the radiolabel is coupled to the protein/peptide through the C-terminal glycine-cysteine tail. In another embodiment, the protein/peptide comprises an N-terminal glycine-cysteine tail and wherein the radiolabel is coupled to the protein/peptide through the N-terminal glycine-cysteine tail. The protein/peptide may be an antibody, for example, a single domain antibody fragment. The protein/peptide is a tumor targeting protein/peptide.

In an embodiment, a method of performing molecular imaging in a subject, comprises administering to the subject an effective amount of a radiolabeled protein/peptide of any of compounds (V)—(VIII).

In an embodiment, a method of treating cancer in a subject, comprises administering to the subject an effective amount of a radiolabeled protein/peptide of any of compounds (V)—(VIII).

In an embodiment, a method of labeling a protein or a peptide with a radioactive halogen comprises:
(i) obtaining a compound having the structure (II):

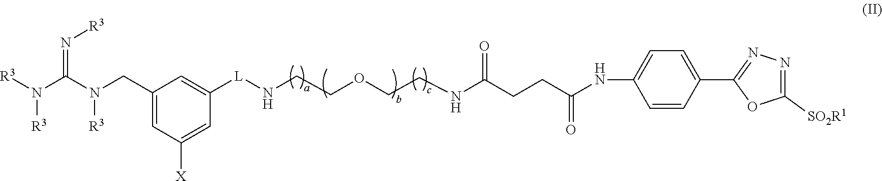

where:
a is 1-6; b is 1-6; c is 1-6;
X is a radiohalogen;
R¹ is C₁₋₆ alkyl;
each R³ is independently a carbamate protecting group or H; and
L is C(O) or

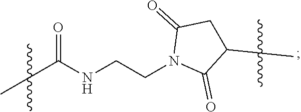 [SMILES: *C(=O)NCCN1C(=O)CC(*)C1=O]

and
(ii) when R³ is H, reacting the compound (II) with the protein or the peptide, or when R³ is a carbamate protecting group, reacting compound (II) with the peptide.

In an embodiment, the method of labeling a protein/peptide with a radioactive halogen further comprises removing carbamate protecting groups (e.g., Boc groups) from the compound prior to reacting the compound with the protein. In an embodiment, the protein or the peptide is an antibody fragment, for example, a single domain antibody fragment. In an embodiment, the protein/peptide is a tumor targeting protein/peptide.

In an embodiment of the present disclosure, the method further comprises modifying a protein/peptide by adding a C-terminal glycine-cysteine tail to the protein/peptide. Exemplary glycine-cysteine tails have the structure GₙC, where n is an integer between 2 and 10. The radiohalogen- or radiohalogen support precursor—prosthetic agent reacts with the cysteine residue of the glycine-cysteine tail to form a thioether bond between the prosthetic agent and the protein/peptide. In another embodiment, the protein/peptide comprises an N-terminal glycine-cysteine tail and wherein the radiolabel is coupled to the protein/peptide through the N-terminal glycine-cysteine tail.

In an embodiment, a radiolabeled protein/peptide made by this method has the structure (VII):

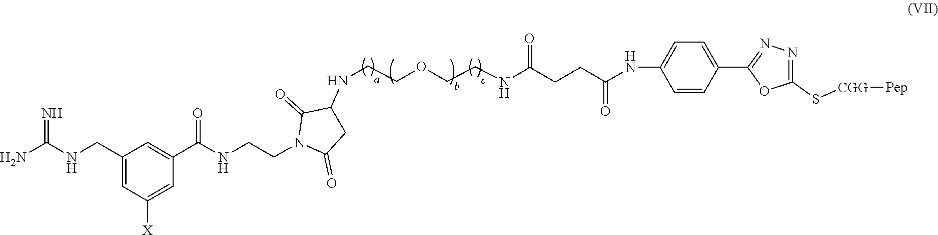

where Pep is a protein/peptide.

In an embodiment, a radiolabeled protein/peptide made by this method has the structure (VIII):

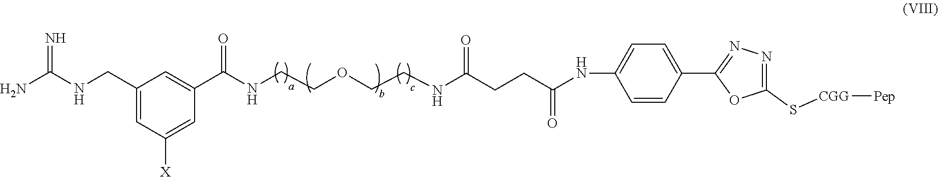

where Pep is a protein/peptide.

### BRIEF DESCRIPTION OF THE DRAWINGS

So that the manner in which the features of the present disclosure can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale; emphasis generally being placed upon illustrating the features of certain embodiments of the invention. Thus, for further understanding of the invention, reference can be made to the following detailed description, read in connection with the drawings in which:

FIG. 1 shows conjugation of a thiol group in a protein or peptide with a radiolabeled moiety-bearing maleimide derivative and thiol exchange reaction of the product with endogenous thiols.

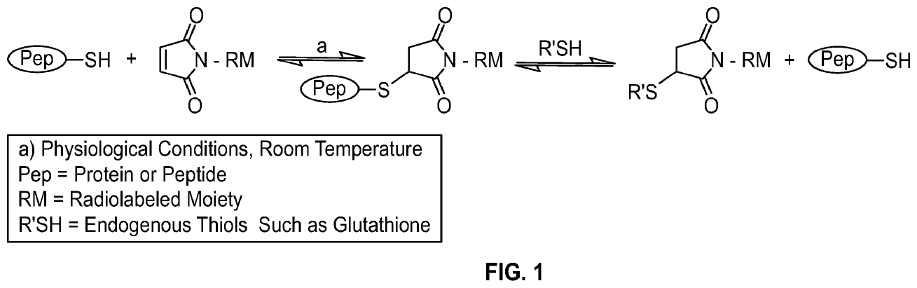

FIG. 2 shows conjugation of 5F7GGC to iso-[¹³¹I]GMIB-PODS and iso-[²¹¹At]AGMB-PODS.

FIG. 3 shows the scheme for the synthesis of [¹⁷⁷Lu]Lu-DOTA-PODS and [¹⁷⁷Lu]Lu-DOTA-PODS-5F7GGC.

FIG. 4 shows data from surface plasmon resonance (SPR) assays used to determine the binding affinities (K_{d}) of 5F7GGC and 5F7GGC conjugates for HER2 extracellular domain.

FIG. 5A shows data from saturation binding assays, using HER2-expressing BT474 human breast carcinoma cells, for the determination of binding affinity of iso-[¹³¹I]GMIB-PODS-5F7GGC.

FIG. 5B shows data from saturation binding assays for iso-[¹³¹I]GMIB-PODS-5F7GGC measured in SKOV-3 cells.

FIG. 5C shows data from saturation binding assays for iso-[²¹¹At]AGMB-PODS-5F7GGC measured in BT474 cells.

FIGS. 6A and 6B shows the results from an in vitro paired-label internalization assay on HER2-positive BT474 breast carcinoma cells co-incubated with [¹²⁵I]MEGMIB-5F7GGC and iso-[¹³¹I]GMIB-PODS-5F7GGC. Data shown are surface-bound (A) and internalized fraction (B), of the radioactivity initially bound to cells after a 1-h incubation at 4° C.

FIGS. 7A and 7B shows the results from an in vitro paired-label internalization assay on HER2-positive BT474 breast carcinoma cells co-incubated with iso-[¹³¹I]GMIB-PODS-5F7GGC and iso-[²¹¹At]AGMB-PODS-5F7GGC. Data shown are surface-bound (A) and internalized fraction (B), respectively, of the radioactivity initially bound to cells after a 1-h incubation at 4° C.

### DETAILED DESCRIPTION OF THE INVENTION

The present disclosure provides novel precursors for radiohalogenation, processes for preparing the radiohalogenated prosthetic agents, and processes for using the radiohalogenated prosthetic agents to prepare a radiolabeled protein or peptide. While it is known that radiohalogenated proteins and peptides can be used for molecular imaging and targeted radiotherapy, common techniques used to label proteins and peptides suffer from a lack of selectivity and/or a lack of stability when administered to a subject. The present disclosure overcomes these problems by using a radiohalogenated prosthetic agent that can couple with a protein/peptide to selectively form a thioether bond with a cysteine residue on the protein. Radiolabeled protein conjugates made with the described radiolabeled prosthetic agents displayed high in vitro stability, high internalization and retention in tumor cells in vitro and excellent tumor uptake in vivo.

### I. Peptides/Proteins

The term “peptide” as used herein refers to chains of amino acids linked together by amide bonds. Examples of peptides include oligopeptides and polypeptides. The term “oligopeptides” as used herein, refers to peptides that are composed of less than 15 amino acids. The term “polypeptide” as used herein, refers to peptides that are composed of 15 or more amino acids. The term “proteins” as used herein, refers to a polypeptide that is composed of 50 or more amino acids.

In a preferred embodiment, the protein/peptide used to form a radiolabeled protein/peptide conjugate is a tumor targeting protein/peptide. As used herein, a “tumor targeting protein/peptide” is a protein/peptide that binds to a target molecule on tumor cells, including a target molecule overexpressed in tumor cells (e.g., expressed to a measurably increased level in tumor cells as compared to normal cells) and/or a target molecule specifically expressed in tumor cells (e.g., substantially not expressed in normal cells). Thus, a tumor targeting protein/peptide may bind to a tumor-associated antigen or receptor and/or to a tumor-specific antigen or receptor. A “tumor-associated antigen or receptor” is an antigen or receptor that is found at elevated levels in tumor cells, but that may also be expressed at lower levels in non-tumor cells. A “tumor-specific antigen or receptor” is an antigen or receptor that is only found, or mostly found, in cancer cells. Numerous tumor targeting proteins/peptides, tumor-associated antigens and receptors, and tumor-specific antigens and receptors are known in the art and routinely used.

A tumor targeting protein/peptide that “specifically binds” or “preferentially binds” to a tumor or to a cancer cell is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A tumor targeting protein/peptide “specifically binds” or “preferentially binds” to a target or antigen if it binds with greater affinity, avidity, more readily, and/or with greater duration than it binds to other molecules. It is also understood that a tumor targeting protein/peptide that specifically or preferentially binds to a first target may or may not specifically or preferentially bind to a second target. As such, “specific binding” or “preferential binding” does not necessarily require (although it can include) exclusive binding.

Preferably, tumor targeting agents (TTAs) used in the invention have high binding affinity, for example, having a dissociation constant K_{D} (k_{off}/kₒₙ) of about 10⁻⁹ M or less.

Representative tumor targeting proteins include antibodies. “Antibodies” are immunoglobulin molecules that recognize and bind to a specific target or antigen, such as a carbohydrate, polynucleotide, lipid, polypeptide, etc., through at least one antigen recognition site, located in the variable region of the immunoglobulin molecule. As used herein, the terms “antibody” and “antibodies” encompass any type of antibody, including but not limited to canonical antibodies, monoclonal antibodies, polyclonal antibodies, bispecific antibodies, multispecific antibodies, heteroconjugate antibodies, recombinantly produced antibodies, humanized antibodies, chimeric antibodies, monovalent antibodies, multivalent antibodies, anti-idiotypic antibodies, antibody fragments (described further below), and fusion proteins having an antibody or antigen-binding fragment thereof, maxibodies, minibodies, intrabodies, diabodies, triabodies, tetrabodies, and any other modified configuration of the immunoglobulin molecule including glycosylation variants of antibodies, amino acid sequence variants of antibodies, and covalently modified antibodies.

Canonical antibodies comprise two heavy (H) and two light (L) polypeptide chains held together by covalent disulfide bonds and non-covalent interactions. Each light chain is composed of one variable domain (VL) and one constant domain (CL). Light chains of the antibodies from any vertebrate species can be assigned to one of two clearly distinct types, called kappa (κ) and lambda (λ), based on the amino acid sequences of their constant domains. Each heavy chain comprises one variable domain (VH) and a constant region, which in the case of IgG, IgA, and IgD antibodies, comprises three domains termed CH1, CH2, and CH3 (IgM and IgE have a fourth domain, CH4).

An “antibody fragment” comprises at least a portion of an intact antibody sufficient to function as a targeting agent as described herein. An antibody fragment generally includes an “antigen-binding fragment” which refers to a polypeptide fragment of an immunoglobulin or antibody that specifically binds or reacts with a selected antigen, target, or immunogenic determinant thereof, or that competes with the intact antibody from which the fragments were derived for specific antigen binding. Antibody fragments include, but are not limited to, a Fab fragment, a Fab′ fragment, a F(ab′)₂ fragment, a Fd fragment, a Fv fragment, a Fc fragment, a scFv fragment, Fc fusions including nanobody-Fc fusions, a dual variable domain (DVD) Fab, single chain antibodies, single domain antibodies (sdAbs, also known as nanobodies and VHH antibodies, for example, VNAR antibodies). In a preferred aspect of the invention, the tumor targeting protein is a single domain antibody fragment (sdAb) also known as a VHH molecule or nanobody.

In one aspect, the antibody is a “multispecific antibody” that binds to more than one antigen or epitope. In some cases, the multispecific antibody binds to two, three, four, five or more target antigens or epitopes. The target antigens or epitopes can be on the same cell or on separate cells. The target antigens can be two or more separate and unique antigens or can be different epitopes of the same antigen. A “bispecific” antibody or antibody fragment binds to two target antigens or epitopes.

In certain aspects, antibodies useful in the invention are fully human antibodies, humanized antibodies, or chimeric antibodies.

In other aspects, antibodies useful in the invention are obtained from camelid species such as dromedary camels, Bactrian camels, wild Bactrian camels, llamas, alpacas, vicunas, and guanacos. In particular, antibodies produced in camelid species have only three hypervariable regions (CDRs), compared to antibody formats derived from conventional IgGs, and as such, these binders preferably recognize and bind conformational epitopes, such as those formed by enzymatic pockets of regulatory domains.

As used herein, the term “antibody” or “antibodies” further encompasses antibody mimetics, i.e., synthetic proteins that show high affinity specific binding similar to antibodies, for example, DARPins, affibodies, knottins, affilins, affimers, affitins, alphabodies, anticalins, avimers, fynomers, kunitx domain peptides, monobodies, nanoCLAMPs, etc.

Representative tumor antigens/receptors to which tumor targeting proteins/peptides useful in the invention bind include, for example, A33, αvβ3, AFP, AKAP-4, ALK, AR, B7-DC (PD-L2), B7H3, B7-H3, BCMA, BCR-ABL, BRCA mutation, BORIS, Clorfl86, CA9, CA-125, CA19-9, CA6, CAIX, CAMPATH-1, CEA, CD19, CD20, CD25, CD30, CD33, CD37, CD45, CD5, CLDN16, CLDN6, CLDN18.2, CMET, CS-1, CCNB1, CXCR2, CYP1B1, DLL3, EGF, EGFR, EGFRvIII, (de2-7 EGFR), EMR2, ENG, EPCAM, EPHA2, ERG, ETV6-AML, EWSRI, FAP, FBP, folate receptor, FOSL1, FRA, FucGMI, G250, GAGE, GD2, GD3, GloboH, GLP-3, GM2, gp100, GPC3, GRP94 (Endoplasmin), HER2, Her-2/neu, HER3, HLA-DR, HMWMAA, HPV E6, HPV E7, hTERT, IL-2 receptor, LCK, LGMN, LewisY, LIV1, LMP2, LRRC15, LY6E, MAD-CT-1, MAD-CT-2, MAGE A1, MAGE A4, MAGE C₂, MAGE-A3, MelanA/MART1, MSLN, mesothelin, ML-IAP, MMPs, MUC1, MUC15, MUC16, MYCN, NA17, NAPI2B, NY—BR-1, NY-ESO-1, OY-TES1, p53 mutant, p53 nonmutant, PAGE4, PAP, PARPs (e.g., PARP-1), PAX3, PAX5, PDGFR-B, PD-L, PD-L1, PLAV1, polySia, PRi, PSA, PSCA, PSMA, PTK7, RAS mutations, RGS5, RhoC, RON, ROR1, ROR2, SART3, sLe(animal), somatostatin receptor, SP17, SSX2, STn, STRA6, tenascin, TEM1, Tie 3, TIM-3, TMEM238, TMPRSS3, TMPRSS4, RAIL1, TROP2, TRP-2, UPK1B, VEGFR1, VEGFR2, VISTA, VTCN1 (B7-H4), WTi, XAGE 1, and TAG-72.

Examples of proteins/peptides that can be coupled to the radiohalogenated prosthetic agent include, but are not limited to, antibodies that bind to molecules that are expressed by cancer cells. In an embodiment, the antibodies are selective for HER2 expressing cancers. In one embodiment, the protein/peptide targeting agent is anti-HER2 5F7 sdAb as described in the publication to Pruszynski et al. “Targeting Breast Carcinoma with Radioiodinated anti-HER2 Nanobody” Nuclear Medicine and Biology 40 (2013) 52-59. Anti-HER2 5F7 sdAb can be modified for coupling to the radiohalogenated prosthetic agent by modifying the anti-HER2 5F7 sdAb to have a C-terminus glycine-cysteine tail. Additional anit-HER2 single domain antibodies are described in U.S. Pat. No. 10,174,117 to Baty et al. and PCT Publication No. WO 2022/152862 to Perez et al., both of which are incorporated herein by reference. Anti-HER2 nanobodies are described in U.S. Patent Application Publication No. 2020/0306392 to Ting et al., which is incorporated herein by reference. Modified Anti-HER2 sdAbs can also be used. For example, VHH_1028 is a modified anti-HER2 sdAb which has been modified to ensure no lysine residues were present in its CDR loops as described in the publication to Feng et al. “Evaluation of an ¹³¹I-labeled HER2-specific single domain antibody fragment for the radiopharmaceutical therapy of HER2-expressing cancers” Sci Rep 12, 3020 (2022) which is incorporated herein by reference.

In some aspects of the invention, the tumor targeting protein is “internalizing,” i.e., it is taken up by the cell, along with a radiolabel bound to the protein or a fragment thereof, upon binding to the target antigen or receptor. As is understood in the art, antibodies may be engineered to be internalizing, or otherwise be selected for this property. See e.g., Zhou et al., Arch Biochem Biophys, 2012, 15; 526(2):107-13.

If the tumor targeting protein/peptide is not naturally internalizing, one or more cell penetration agents may be coupled to the tumor targeting protein/peptide to promote intracellular delivery of the radiolabeled protein/peptide. Cell penetration agents can protect the radiolabeled protein/peptide from endosomal entrapment and/or lysosomal degradation.

Representative cell penetration agents include cell-penetrating peptides (CPPs) that are typically 10 to 30 amino acid (aa) peptides in length and are either arginine-rich and amphipathic, or lysine-rich and hydrophobic. A CPP can be, for example, a cationic peptide, amphipathic peptide or hydrophobic peptide, e.g. consisting primarily of Tyr, Trp and Phe, dendrimer peptide, constrained peptide or crosslinked peptide. See e.g., Herce et al, Nat Chem, 2017, 9:762-771.

In some aspects of the invention, a nuclear localization peptide (NLP) may be additionally or alternatively used to promote nuclear localization of the radiolabeled protein/peptide. For example, karyophilic peptides, composed of at least four arginines, (R), and lysines, (K), within a hexapeptide flanked by proline and glycine helix-breakers, can be used as NLPs for radiolabeled peptides. See e.g., Chen et al, J Nucl Med, 2006, 47: 827-836.

### II. Radiohalogenated Prosthetic Agent

One aspect of the present disclosure provides a radiohalogenated prosthetic agent having the structure (I).

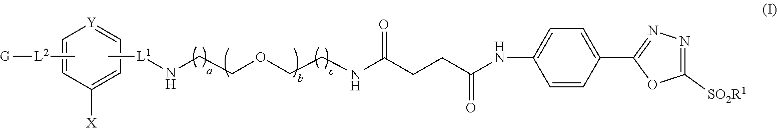

where:
a is 1-6; b is 1-6; c is 1-6;
L¹ is —C(O)— or

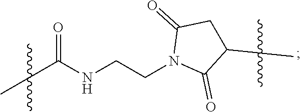 [SMILES: *C(=O)NCCN1C(=O)CC(*)C1=O]

L² is a bond or —(CH₂)ₙ—, where n is 1-6;
G is guanidine, a guanidine having one or more carbamate protecting groups, PO₃H, SO₃H, PO₂-OtBu, SO₂—OtBu, arginine, phosphono-phenylalanine, sulfo-phenylalanine, glutamate, aspartate, lysine, a hydrophilic carbohydrate moiety, or a polyethylene glycol (PEG) chain;
Y is CH or N;
X is a radiohalogen; and
R¹ is C₁₋₆ alkyl.

The terminal guanidine group can be unprotected or protected with one or more carbamate protecting groups. Exemplary carbamate protecting groups include, but are not limited to, tert-butyloxycarbonyl (“Boc”), allyloxycarbonyl (“Alloc”), fluorenylmethyloxycarbonyl (“Fmoc”), 2-(trimethylsilyl)ethoxycarbonyl (“TeoC”), and carboxybenzyl (“CBz”). A guanidine moiety can be protected by one, two, three or four carbamate protecting groups. In a specific embodiment the guanidine is a Boc protected guanidine such as mono Boc-protected guanidine, di Boc-protected guanidine, tri Boc-protected guanidine, or tetra Boc-protected guanidine.

In one embodiment, a radiohalogenated prosthetic agent has the structure (II).

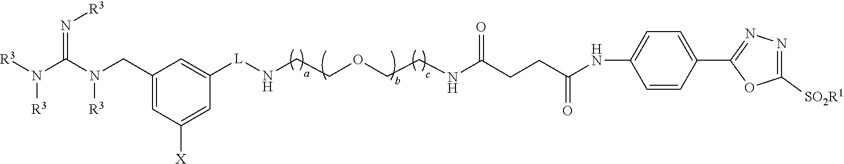

where:
a is 1-6; b is 1-6; c is 1-6;
L is —C(O)— or

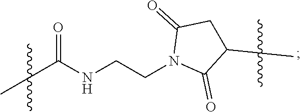 [SMILES: *C(=O)NCCN1C(=O)CC(*)C1=O]

X is a radiohalogen;
R¹ is C₁₋₆ alkyl;
each R² is independently C₁₋₆ alkyl; and
each R³ is independently a carbamate protecting group or H.

In some specific embodiments, a radiohalogenated prosthetic agent has the structure (II), where R¹ is methyl, ethyl, or propyl and where a is 1 or 2; b is 2 or 3; and c is 1 or 2.

Radiohalogens that can be used include, but are not limited to, ¹⁸F, ¹²²I, ¹²³I, ¹²⁴I, ¹²⁵I, ¹³¹I, ⁷⁵Br, ⁷⁶Br, ⁷⁷Br, ^{80m}Br, and ²¹¹At.

A specific example of a radiohalogenated prosthetic agent is the compound having the structure (III).

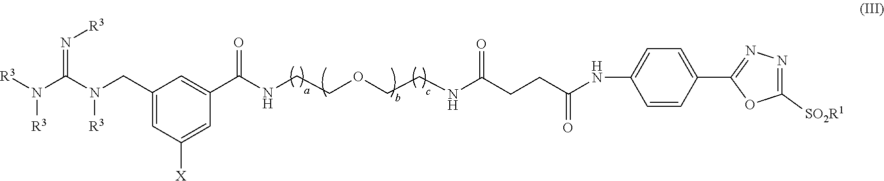

where:
a is 1-6; b is 1-6; c is 1-6;
X is a radiohalogen;
R¹ is C₁₋₆ alkyl;
each R² is independently C₁₋₆ alkyl;
each R³ is independently a carbamate protecting group or H.

Another specific example of a radiohalogenated prosthetic agent is the compound having the structure (IV).

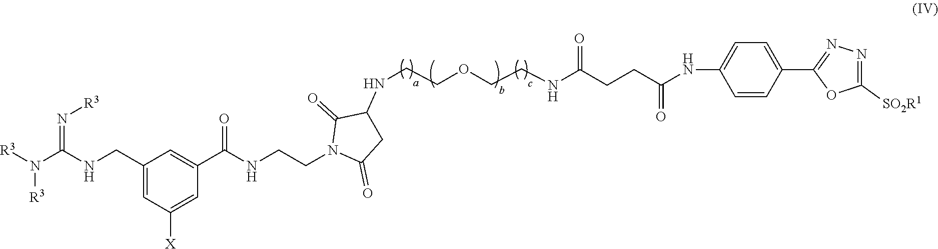

where:
a is 1-6; b is 1-6; c is 1-6;
X is a radiohalogen;
R¹ is C₁₋₆ alkyl;
each R² is independently C₁₋₆ alkyl;
each R³ is independently a carbamate protecting group or H.

### III. Protein/Peptide—Radiohalogenated Prosthetic Agent Conjugates

In an embodiment, the radiohalogenated prosthetic agent described herein can be covalently linked to a protein/peptide to form a radiolabeled protein/peptide. In one embodiment, a radiolabeled protein/peptide includes one or more radiohalogenated prosthetic agents coupled to a protein/peptide through a thioether bond, wherein the radiolabeled protein/peptide has structure (V), or the radiolabeled protein/peptide is a pharmaceutically acceptable salt of structure (V):

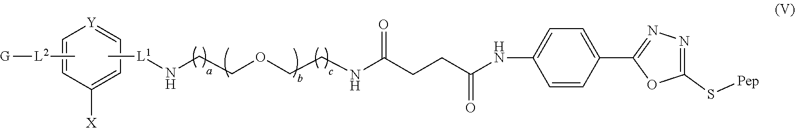

where:
a is 1-6; b is 1-6; c is 1-6;
L is C(O) or

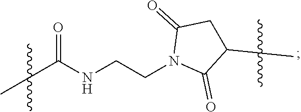 [SMILES: *C(=O)NCCN1C(=O)CC(*)C1=O]

L² is a bond or —(CH₂)ₙ—, where n is 1-6;
G is guanidine, a guanidine having one or more carbamate protecting groups, PO₃H, SO₃H, PO₂—OtBu, SO₂—OtBu, arginine, phosphono-phenylalanine, sulfo-phenylalanine, glutamate, aspartate, lysine, a hydrophilic carbohydrate moiety, or a polyethylene glycol (PEG) chain;
Y is CH or N;
X is a radiohalogen;
R¹ is C₁₋₆ alkyl; and
Pep is a protein/peptide.

In a specific embodiment, a radiolabeled protein/peptide has the structure (VI), or the radiolabeled protein/peptide is a pharmaceutically acceptable salt of structure (VI).

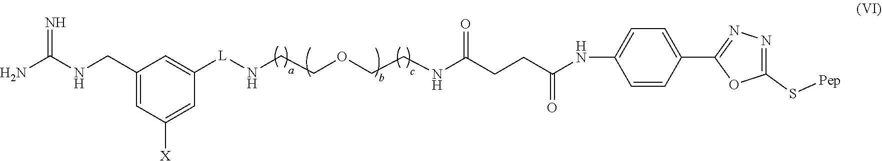

where:
a is 1-6; b is 1-6; c is 1-6;
X is a radiohalogen;
L is C(O) or

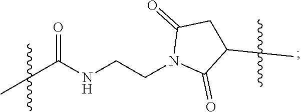 [SMILES: *C(=O)NCCN1C(=O)CC(*)C1=O]

and
Pep represents a protein/peptide.

In an embodiment, the radiohalogen is selected from the group consisting of ¹⁸F, ¹²²I, ¹²³I, ¹²⁴I, ¹²⁵I, ¹³¹I, ⁷⁵Br, ⁷⁶Br, ⁷⁷Br, ^{80m}Br, and ²¹¹At.

In an embodiment, the protein/peptide comprises at least one cysteine residue and wherein one or more of the radiohalogenated prosthetic agents are coupled to the protein/peptide to the cysteine residue.

In specific embodiments, a protein/peptide may be an antibody. For example, the protein/peptide may be a single domain antibody fragment. In specific embodiments, the protein/peptide is a tumor targeting protein.

In some embodiments, a protein/peptide may be modified prior to coupling with the radiolabel by incorporating one or more cysteine amino acids into the protein/peptide. In one embodiment, at least one cysteine is incorporated onto the C-terminal end of the protein/peptide. For example, a glycine-cysteine tail may be added to the C-terminal end of the protein/peptide. A method of incorporating a C-terminal cysteine group on a protein is discussed in Pruszynski et al. “Targeting breast carcinoma with radioiodinated anti-HER2 nanobody” Nuclear Medicine and Biology 40 (2013) 52-59, which is incorporated herein by reference.

In an embodiment, a specific example of a radiohalogenated-protein/peptide is the compound having the structure (VII):

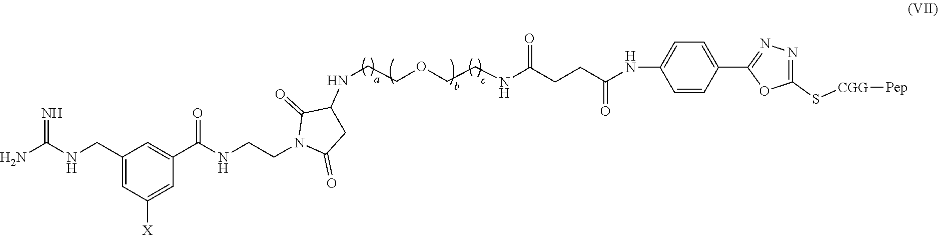

where Pep is a protein/peptide.

In another embodiment, a specific example of a radiohalogenated-protein/peptide is the compound having the structure (VIII):

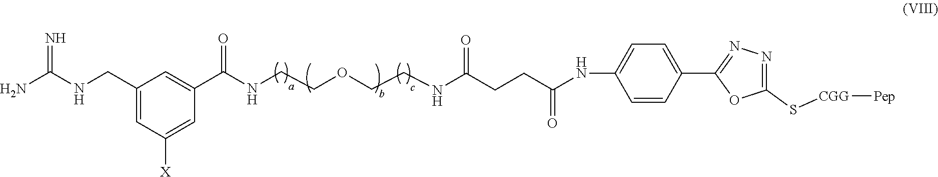

where Pep is a protein/peptide.

### IV. Synthesis of Radiohalogenated Prosthetic Agents

The radiohalogenated prosthetic agents described herein can be synthesized by coupling a phenyloxadiazolyl methyl sulfone (PODS) containing linker to a radiolabel support compound. The PODS linker includes a phenyloxadiazolyl methyl sulfone that is used to selectively form a thioether bond with a cysteine in a protein/peptide. The phenyloxadiazolyl methyl sulfone is situated at one end of the molecule. The remainder of the PODS linker comprises a linker group that is used to couple the phenyloxadiazolyl methyl sulfone to a radiolabel support. The PODS linker has the general structure (IX):

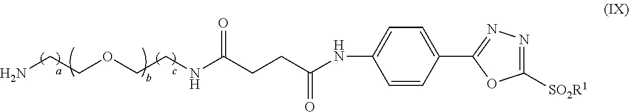

where:
a is 1-6; b is 1-6; c is 1-6; and
R¹ is C₁₋₆ alkyl.

In a preferred embodiment, the PODS linker has the structure (IX), where: a is 1 or 2; b is 2 or 3; c is 1 or 2; and R¹ is methyl, ethyl, or propyl. An exemplary PODS linker (known hereinafter as “PODS”) is depicted below (X).

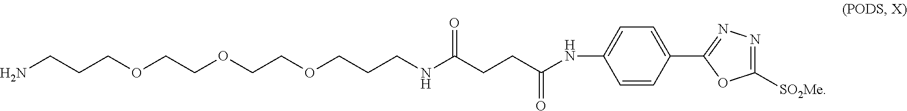 [SMILES: CS(=O)(=O)c1nnc(-c2ccc(NC(=O)CCC(=O)NCCCOCCOCCOCCCN)cc2)o1]

Methods of preparing a PODS linker are taught in U.S. Pat. No. 11,000,604, which is incorporated herein by reference.

The terminal amine (—NH₂) of the PODS linker is coupled to a radiolabel support compound. In one embodiment, the radiolabel support compound has the structure (XI):

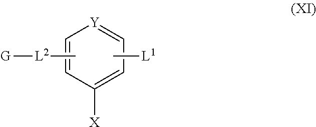

where: L¹ is

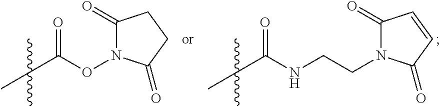 [SMILES: *C(=O)ON1C(=O)CCC1=O ; *C(=O)NCCN1C(=O)C=CC1=O]

L² is a bond or —(CH₂)ₙ—, where n is 1-6;
G is guanidine, a guanidine having one or more carbamate protecting groups, PO₃H, SO₃H, PO₂—OtBu, SO₂—OtBu, arginine, phosphono-phenylalanine, sulfo-phenylalanine, glutamate, aspartate, lysine, a hydrophilic carbohydrate moiety, or a polyethylene glycol (PEG) chain;
Y is CH or N;
X is SnR²₃, B(OH)₂, Bpin, or a radiohalogen;
each R² is independently C₁₋₆ alkyl.

In a specific embodiment, a radiolabel support compound has the structure (XII):

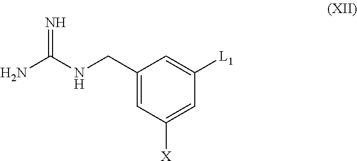

where:
L¹ is

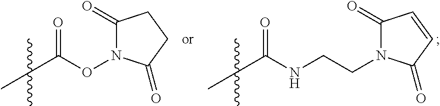 [SMILES: *C(=O)ON1C(=O)CCC1=O ; *C(=O)NCCN1C(=O)C=CC1=O]

X is SnR²₃, B(OH)₂, Bpin, or a radiohalogen; and
each R² is independently C₁₋₆ alkyl.

Specific radiolabel support compounds include, but are not limited to: N-succinimidyl 3-guanidinomethyl-5-(trimethylstannyl)benzoate (iso-SGMTB, XIII); N-succinimidyl 3-guanidinomethyl-5-[*I]iodobenzoate (iso-[*I]SGMIB, XIV); N-succinimidyl 3-[²¹¹At]astato 5-guanidinomethyl benzoate (iso[²¹¹At]SAGMB, XV); N-maleimidoethyl 3-guanidinomethyl-5-(trimethylstannyl)benzamide (MEGMTB, XVI); N-maleimidoethyl 3-[²¹¹At]astato-5-guanidinomethylbenzamide (MEAGMB, XVII) and N-maleimidoethyl 3-guanSAGMBidinomethyl-5-iodobenzamide (MEGMIB, XVIII).

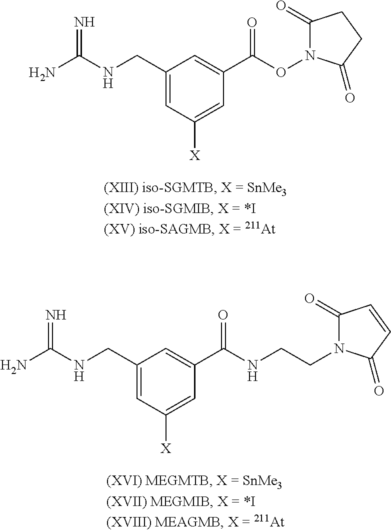

Where *1 is an isotope of iodine including, but not limited to, stable (127) or radioactive (131, 123, 125, and other) isotopes of iodine.

Methods of preparing radiolabel support compounds are taught in U.S. Patent Application Publication No. 2020/0188541, which is incorporated herein by reference.

The PODS linker is coupled to the radiohalogen support precursor to form the radiohalogenation support precursor-PODS conjugate. When the support has a maleimide group, the general reaction for coupling is shown in Scheme (1).

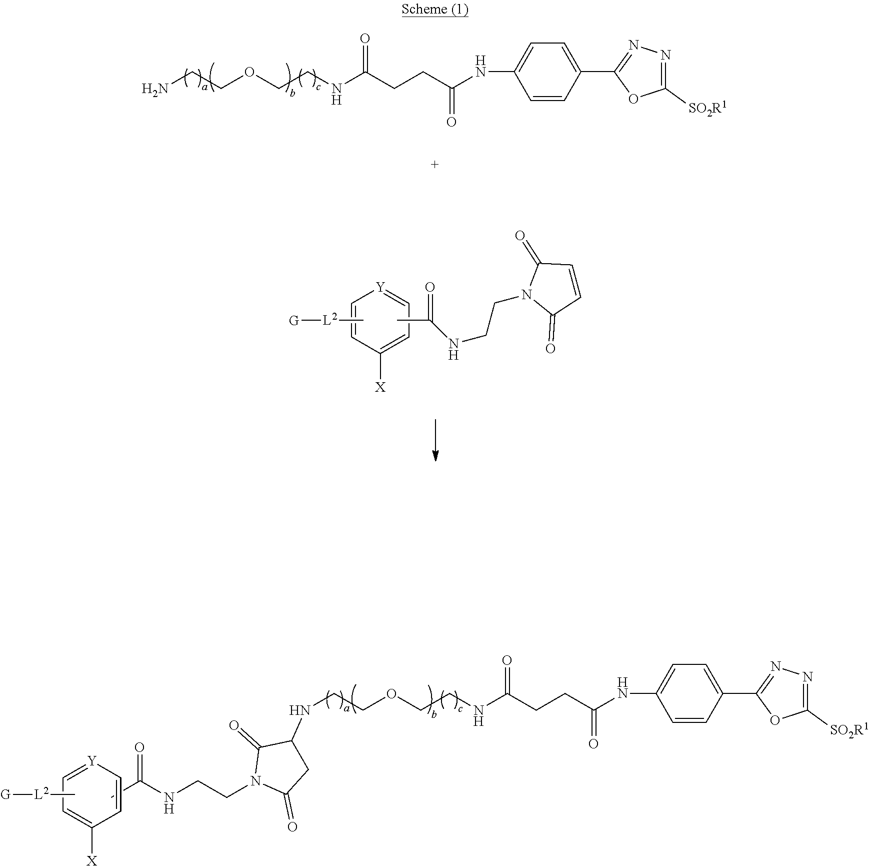

The coupling of the terminal amine of the PODS linker with the maleimide group of the radiohalogen support precursor is performed under basic conditions. Typical basic conditions include reaction in the presence of a tertiary amine such as trimethylamine or diisopropylethylamine. In other embodiments, a buffered aqueous solution (pH>7) can be used as the reaction medium.

With the active ester N-hydroxysuccinimide, the general reaction for coupling is shown in Scheme (2).

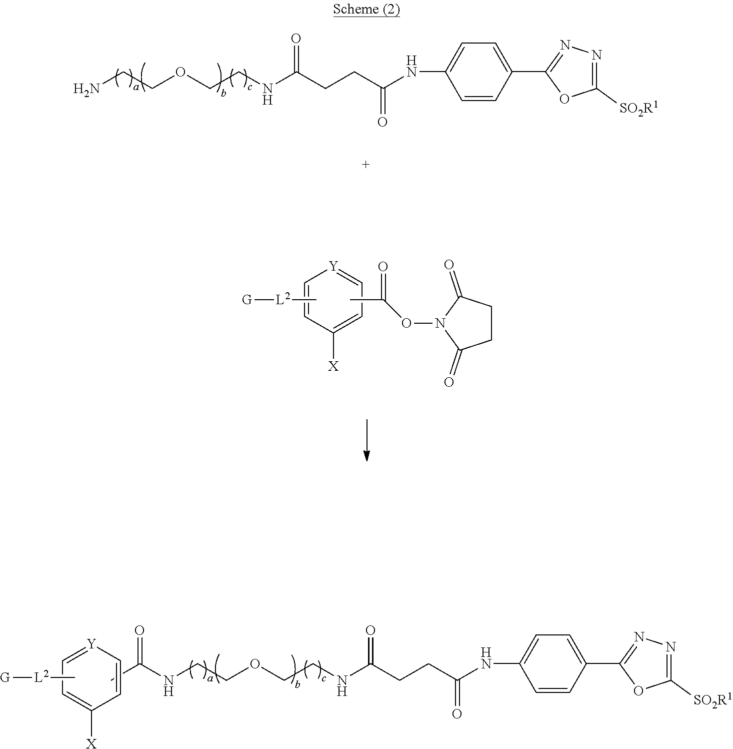

The coupling of the terminal amine of the PODS linker with the N-hydroxysuccinimide active ester group of the radiohalogen support precursor is performed under basic conditions, similar to Reaction Scheme (1). Typical basic conditions include reaction in the presence of a tertiary amine such as trimethylamine or diisopropylethylamine. In other embodiments, a buffered aqueous solution (pH>7) can be used as the reaction medium.

Details regarding similar coupling reactions between N-hydroxysuccinimide active ester and maleimide with amines can be found in U.S. Patent Application Publication No. 2020/0188541, which is incorporated herein by reference.

In an embodiment of the present disclosure, the final step in preparing the radiohalogenated prosthetic agent is the conversion of the trialkyl tin or boronate groups to the radiohalogen. Generally, a trialkyltin-substituted aromatic compound (e.g., present in the precursor of the radiohalogen support) is reacted with a radiohalogen in the presence of an oxidizing agent. The oxidizing agent oxidizes the radiohalogen, allowing substitution of the trialkyl tin moiety by the radiohalogen to occur. The process is performed in a suitable solvent or solvent system for dissolving the reactants. Scheme (3) shows this process for the amide-bearing PODS-radiohalogen support. A similar process is used to transform a trialkyl tin-substituted, maleimide coupled PODS precursor to a radiohalogenated support. In a preferred embodiment, the oxidizing agent is N-chlorosuccinidimide. Methods of forming radiohalogenated compounds are discussed in the article to Dubost et al. “Recent Advances in Synthetic Methods for Radioiodination”, J. Org. Chem. 2020, 85, 8300-8310, which is incorporated herein by reference. Conversion of boronic acid and ester precursors to radiohalogenated derivatives is typically performed with nucleophilic radiohalide and this conversion does not need an oxidizing agent (Kondo et al., Journal of Labelled Compounds and Radiopharmaceuticals (2021), 64(8), 336-345; Reilly et al., Organic Letters (2018), 20(7), 1752-1755).

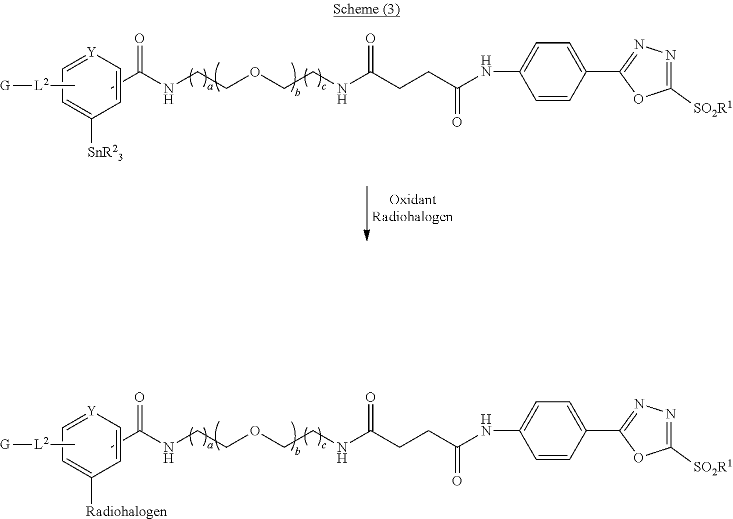

### V. Synthesis of Radiolabeled Proteins and Peptides

The present disclosure further describes methods of radiolabeling a protein/peptide (e.g., methods of radiolabeling a tumor targeting protein). The method is generally shown below in reaction Scheme (4).

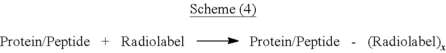

where x is at least one.
x is related to the number of groups present on the protein/peptide, that can react with the radiohalogenated prosthetic agent.

In one embodiment, an oxadiazolyl sulfone-based coupling methodology can be used to couple a radiolabel to a protein/peptide. This coupling methodology is shown in Scheme (5). In this reaction (Reaction Scheme (5)) an oxadiazolyl sulfone-reagent selectively reacts with thiols to form a stable protein/peptide-radiolabel conjugate.

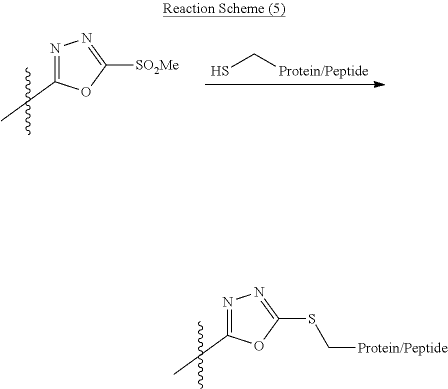

The reaction typically is run by mixing the protein/peptide with the radiohalogenated prosthetic agent. In some embodiments, the peptide is reacted with a reducing agent to break any disulfide bonds prior to reaction with the radiohalogenated prosthetic agent. In an embodiment, the reducing agent is tris(2-carboxyethyl)phosphine (“TCEP”) or dithiothreitol (DTT). Scheme (6) shows an example of conjugation of the radiolabels of the present disclosure with a protein/peptide. In the reaction depicted, a tail group comprising a pendant cysteine group has been appended to the protein/peptide of interest. It should be understood, however, that the same reaction can be performed on a cysteine amino acid that is present in the unmodified protein/peptide.

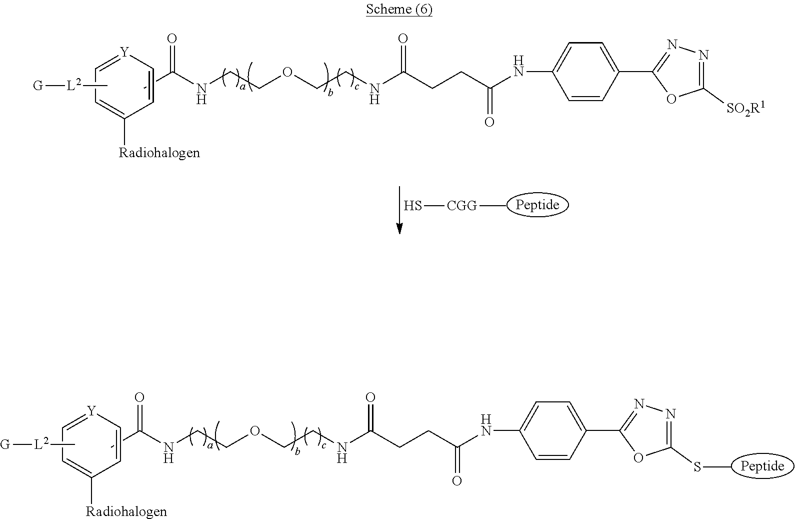

In another embodiment of the present disclosure, the protein/peptide is coupled to the radiohalogenation support precursor, followed by radiolabeling of the protein/peptide-radiohalogenation support precursor. When coupling a protein to the radiohalogenation support, the guanidine protecting groups are preferably removed. The method is generally shown below in reaction Scheme (7).

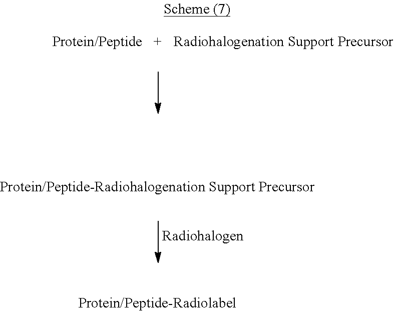

The reactions used in Scheme (7) are similar, if not identical, to the reactions used in the corresponding transformations in the previous reaction schemes.

### VI. Uses

The radiolabeled conjugates prepared as described herein have versatile uses in therapy and imaging, i.e., any use that would benefit from targeted delivery of a radionuclide, such as a radiohalogen.

### A. Therapy Using Radiolabeled Conjugates

It will be appreciated that the radiolabeled protein/peptide conjugates of the present disclosure may be used for treatment of cancer or other neoplastic disorders, whether administered alone or in combination with an additional anti-cancer agent or radiotherapy. The radiohalogenated protein/peptide conjugates disclosed herein may be used to treat any of various cancers and other neoplastic conditions as are recognized in the art, for example solid tumors, such as tumors of the ovary, breast, adrenal, liver, kidney, bladder, gastrointestinal tract, cervix, uterus, prostate, pancreas, lung, thyroid, and brain. The radiohalogenated protein/peptide conjugates disclosed herein may be used to treat hematologic malignancies as well.

Additional neoplastic conditions subject to treatment in accordance with the instant invention may be selected from the group including, but not limited to, adrenal gland tumors, AIDS-associated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma).

In certain embodiments, the radiohalogenated protein/peptide conjugates are used to treat breast cancer, including invasive or infiltrating breast cancer, recurrent breast cancer, and/or refractory breast cancer. In some certain embodiments, the breast cancer is HER2±cancer.

The radiolabeled conjugates disclosed herein may be used as first in class targeted therapies for the treatment of cancer, particularly those cancers characterized by solid tumors. A significant advantage of the disclosed conjugates is optimization of the therapeutic index by reducing or eliminating side effects that occur as a result of nonspecific targeting of a therapeutic radionuclide. As such, the disclosed conjugates may be used at reduced doses and/or less aggressive administration regimens as a result of the improved therapeutic index. Specifically, conjugates as disclosed herein may show improved therapeutic outcomes, including but not limited to reduction of tumor size, delayed tumor growth, fewer metastases, and/or increased longevity. Such improvements are at least about 10%, or at least about 20%, or at least about 30%, or at least about 40%, or at least about 50%, or at least about 60%, or at least about 70%, or at least about 80%, or at least about 90%, or at least about 95%, or at least about 99%, when compared to a therapeutic effect observed in a subject receiving a radiohalogen without protein/peptide targeting. In other aspects, such improvements are at least about 2-fold, or at least about 5-fold, or at least about 10-fold, or at least about 20-fold, or at least about 50-fold, or at least about 100-fold, or more, when compared to any therapeutic effect observed in a subject receiving the radiohalogen only, i.e., without targeting as in a radiolabeled protein/peptide conjugate described herein.

### B. Imaging Using Radiolabeled Conjugates

Subjects may also be administered protein/peptide conjugates as described in the present disclosure for imaging of tumors or other biological features. The radiolabeled protein/peptide conjugates may be prepared with a protein/peptide that targets a specific type of tumor cell, for example. Administration of the protein/peptide conjugate to the subject can localize the radiohalogen at the site of interest. Standard diagnostic imaging techniques for radioactive isotopes can then be used to determine the presence and/or the extent of a specific type of tumor, including monitoring of tumor progression and/or response to treatment, including detection and monitoring for any of the tumor types noted above. In particular aspects, radiolabeled protein/peptide conjugates can be used for diagnostic imaging of breast cancer, including any of the specific types of breast cancer noted herein above, and more specifically, HER2±breast cancer.

### C. Formulation, Administration, and Dose

The disclosed conjugates of the invention may be formulated as desired using art-recognized techniques. In some embodiments, the therapeutic compositions of the invention may be administered neat or with a minimum of additional components while others may optionally be formulated to contain suitable pharmaceutically acceptable carriers (e.g., vehicles, adjuvants, and diluents) comprising excipients and auxiliaries that are well known in the art, including for example, pH adjusting and buffering agents, tonicity adjusting agents, stabilizers, wetting agents, radioprotectants and the like. Certain non-limiting exemplary carriers include saline, buffered saline, dextrose, water, glycerol, ethanol, and combinations thereof. Certain non-limiting exemplary radioprotectants include ascorbic acid, gentisic acid, ethanol and combinations thereof.

In general, the compounds and compositions of the invention may be administered to a subject by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms suitable for the particular mode of administration, including, for example, tablets, capsules, powders, granules, ointments, solutions, suppositories, enemas, injections, inhalants, and aerosols.

The particular dosage regimen for administering conjugates of the invention, i.e., dose, timing and repetition, will depend on the particular subject and that subject's medical history, as well as empirical considerations such as pharmacokinetics (e.g., half-life, clearance rate, etc.). Frequency of administration may be determined and adjusted over the course of therapy. A therapeutically effective dose is a dose sufficient to provide a clinical benefit to the subject, including for example, a dose sufficient to reduce tumor size, maintain a reduction of tumor size, reduce or slow tumor growth, delay the development of metastasis, improve longevity, etc. Dosage administered may be adjusted or attenuated to manage potential side effects and/or toxicity.

As used herein, the term “subject” and “patient” are used interchangeably and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

The compositions provided herein may be used for many in vitro and in vivo uses, including treatment of diseases, imaging of tissues, and the like. For example, in one embodiment the composition may be used for the imaging of a tumor or other tissue which comprises administering to the subject an effective amount of a composition as provided herein.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

In other embodiments, the compositions provided herein are used for the prevention and/or treatment of a disease in a subject. As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition.

A therapeutically effective amount is an amount of the disclosed radiolabeled protein/peptide that results in a desired therapeutic effect, for example, an increase in longevity and/or quality of life. In the context of cancer, desired therapeutic effects also include reduction of tumor size, slowing of tumor growth, decreased or slowing of metastasis, decreased tumorigenicity, decreased or amelioration of symptoms or indicators/biomarkers associated with cancer, etc. A therapeutically effective amount may be administered in a single dose or multiple doses.

For imaging applications, an effective amount is an amount of the disclosed radiolabeled protein/peptide that results in measurable in vivo or ex vivo detection following administration to a patient, with sufficient sensitivity to assess biodistribution of the radiolabeled protein/peptide above any background or nonspecific detection. Any detection technique known in the art may be used, for example, SPECT/CT imaging, PET, PET/CT, and PET/MRI. As for therapeutic applications, an effective amount for imaging applications may be administered in a single dose or multiple doses.

### EXAMPLES

The following examples are included to demonstrate certain embodiments of the invention.

### Materials and Methods

Sodium [¹²⁵I]iodide (629 GBq/mg) in 0.1 M NaOH was obtained from PerkinElmer (Boston, MA). Sodium [¹³¹I]iodide (936 GBq/mg, 236.097 GBq/mL) was obtained from International Isotopes Inc. (Idaho Falls, ID) in a solution of 0.04 M sodium thiosulfate, 0.2 M NaOH and 0.2 M sodium carbonate. All reagents and solvents were purchased from Fisher Scientific unless otherwise stated. Boc₂-iso-SGMTB, Boc₂-iso-SGMIB, Boc₂-MEGMTB, PODS, and PODS-DOTA were synthesized following reported methods (Feng, Y., et al., “Site-specific radioiodination of an anti-HER2 single domain antibody fragment with a residualizing prosthetic agent.” Nuclear Medicine and Biology, 2021. 92: p. 171-183; Adumeau, P., M. Davydova, and B. M. Zeglis, “Thiol-reactive bifunctional chelators for the creation of site-selectively modified radioimmunoconjugates with improved stability.” Bioconjugate Chemistry, 2018. 29(4): p. 1364-1372; and Davydova, M., et al., “Synthesis and bioconjugation of thiol-reactive reagents for the creation of site-selectively modified immunoconjugates.” Journal of Visualized Experiments: JoVE, 2019(145), each of which is incorporated herein by reference). Production, purification and characterization details for anti-HER2 sdAb 5F7GGC have been reported (Feng, Y., et al., “Site-specific radioiodination of an anti-HER2 single domain antibody fragment with a residualizing prosthetic agent.” Nuclear Medicine and Biology, 2021. 92: p. 171-183 and Pruszynski, M., et al., “Targeting breast carcinoma with radioiodinated anti-HER2 Nanobody” Nuclear Medicine and Biology, 2013. 40(1): p. 52-59, both of which are incorporated herein by reference). Synthesis of [^{125/131}I]MEGMIB-5F7GGC was performed following a reported method (Feng, Y., et al., “Site-specific radioiodination of an anti-HER2 single domain antibody fragment with a residualizing prosthetic agent.” Nuclear Medicine and Biology, 2021. 92: p. 171-183). [²¹¹At]MEAGMB-5F7GGC, was synthesized using an essentially identical procedure. The anti-HER2 mAb, trastuzumab (Roche/Genentech), was obtained from the Duke University Medical Center Pharmacy. All instruments were calibrated and maintained according to standard quality control practices and procedures. Reverse-phase HPLC (RP-HPLC) purification was performed on a Shimadzu HPLC system (Shimadzu Scientific Instruments, Kyoto, Japan). Both the analytical (250×2 mm, 5 μm, 300 Å) and preparative (250×10 mm, 5 μm, 300 Å) HPLC columns were purchased from Phenomenex (Jupiter Proteo HPLC columns, Phenomenex, Torrance, CA, USA). HPLC analysis was performed using LabSolutions LC/GC software (Shimadzu Scientific Instruments, Kyoto, Japan). Evaporation of solvents was accomplished with either a vacuum evaporator (Biotage V-10 Touch, V10-2XX, Biotage, Uppsala, Sweden) or a rotary evaporator (Hei-VAP Core, Heidolph, Schwabach, Germany). Gel permeation (GP) HPLC was utilized for purification and identification of the 5F7GGC-prosthetic agent conjugates using an Agilent PL Multisolvent 20 column eluted with pure Milli-Q© water as the mobile phase. Purification of radiolabeled compounds was performed with Agilent 1260 Infinity systems using a reversed-phase HPLC column (Agilent Poroshell 120 C18, 2.7 μm, 4.6×50 mm) eluted at a flow rate of 2 mL/min with a gradient consisting of water (solvent A) and acetonitrile (solvent B), both containing 0.1% TFA; the proportion of B was increased linearly from 40% to 80% over 8 min. This system was equipped with a 1260 Infinity Multiple Wavelength Detector (Santa Clara, CA). For monitoring radioactivity, one system was connected to a Dual Scan-RAM flow activity detector/TLC scanner and the other to a Flow-RAM detector (Lablogic, Tampa, FL); both the HPLC and the gamma detectors were controlled by LabLogic Laura software. A CRC-7 dose calibrator (Capintec, Pittsburgh, PA) was used to measure radioactivity at higher levels and for assessing lower activity levels, either an LKB 1282 (Wallac, Finland) or a Perkin Elmer Wizard II (Shelton, CT) automated gamma counter was used.

### Synthesis of Boc₂-iso-GMTB-PODS (XIX)

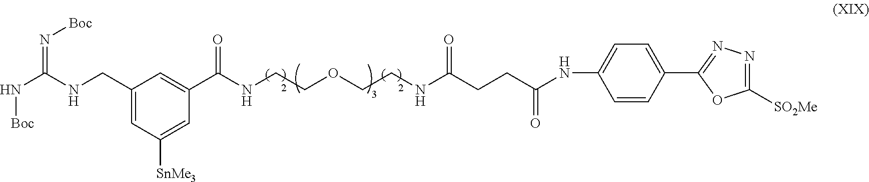

N,N-diisopropylethylamine (DIPEA, 8.0 μL, 45.8 μmol, 3.0 eq.) was added to a solution of PODS (24.8 mg, 45. 8 μmol, 3.0 eq.) in 1.0 mL anhydrous dimethylformamide (DMF). A solution of Boc₂-iso-SGMTB (10 mg, 15.3 μmol, 1.0 eq.) in 1.0 mL DMF was then added to above and the resultant solution was stirred at room temperature overnight protecting it from light. To determine the progress of the reaction, RP-HPLC was performed using the analytical column (1 mL/min, 5-95% acetonitrile in water (0.1% TFA) over 35 min; t_{R} of product: 25.4 min). Once the reaction was deemed complete, the DMF was evaporated completely with a vacuum evaporator. The resultant crude product was re-constituted in 2 mL acetonitrile, and the solution was filtered with a syringe filter. The product was purified via RP-HPLC using a preparative column (6 mL/min, 5-95% acetonitrile in water (0.1% TFA) in 35 min; t_{R}, product: 29.6 min). The purity of isolated sample was assessed using analytical RP-HPLC (1 mL/min, 5-95% acetonitrile in water with 0.1% TFA over 35 min; t_{R} of product: 25.4 min; >95% purity). The solvents from the pooled HPLC fractions containing the product were removed by lyophilization overnight to yield a white powder (4.9 mg, 30.0% yield): ¹H NMR (500 MHz, CDCl₃) δ: 9.90 (s, 1H), 9.50 (s, 1H), 7.99 (d, 2H, J=8.8 Hz), 7.74 (m, 3H), 7.58 (s, 1H), 7.45 (s, 1H), 7.07 (s, 1H), 6.87 (s, 1H), 5.17 (s, 2H), 3.47-3.64 (m, 18H), 3.36 (q, 2H, J=5.9 Hz), 2.66 (m, 2H), 2.57 (m, 2H), 1.90 (quint, 2H, J=6.2 Hz), 1.74 (quint, 2H, J=6.0 Hz), 1.48 (s, 9H), 1.36 (s, 9H), 0.27 (s, 9H). ¹³C NMR (500 MHz, DMSO-d₆) δ: 171.8, 171.5, 167.0, 166.2, 163.2, 162.2, 160.1, 154.5, 144.1, 142.5, 138.5, 137.5, 134.4, 132.9, 129.0, 126.7, 119.6, 116.4, 84.3, 78.3, 70.2, 70.0, 68.7, 68.5, 47.5, 43.4, 37.1, 36.3, 32.2, 30.5, 29.9, 29.8, 28.4, 27.8, 8.8. ESI-MS m z calculated for C₄₅H₆₉N₈O₁₃SSn (M±H)^{±}: 1080.85; found: 1081.0.

### Synthesis of Boc₂-iso-GMIB-PODS (XX)

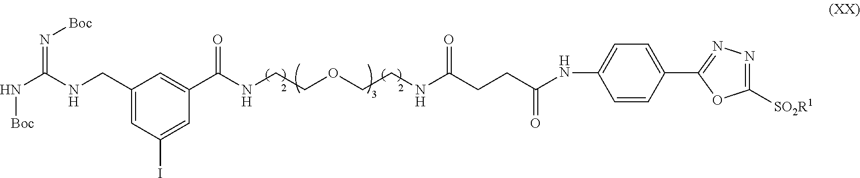

DIPEA (6.4 μL, 36.5 μmol, 3.0 eq.) was added to a solution of PODS (19.7 mg, 36.5 μmol, 3.0 eq.) in 1.0 mL DMF. Next, a solution of Boc₂-iso-SGMIB (7.5 mg, 12.2 μmol, 1.0 eq.) in 1.0 mL of DMF was added to the PODS mixture. The reaction mixture was stirred at room temperature overnight. Progress of the reaction was followed by RP-HPLC, which was performed using the analytical column (1 mL/min, 5-95% acetonitrile in water with 0.1% TFA in 35 min; t_{R} of product: 23.5 min). When the reaction was deemed to be complete, the DMF was evaporated completely with a vacuum evaporator. The resulting crude product was then re-constituted with 2 mL acetonitrile, and the solution was filtered with a syringe filter. The product was purified via RP-HPLC using a preparative column (6 mL/min, 5-95% acetonitrile in water with 0.1% TFA over 35 min; t_{R} of product: 28.0 min). The purity of isolated sample was assessed using analytical RP-HPLC (1 mL/min, 5-95% acetonitrile in water with 0.1% TFA, 35 min; t_{R} of product: 23.5 min; >95% purity). The pooled HPLC fractions containing the product were lyophilized overnight to yield a white powder (8.5 mg, 67.0% yield): ¹H NMR (500 MHz, CDCl₃) δ: 9.75 (s, 1H), 9.5 (s, 1H), 8.00 (d, 2H, J=8.8 Hz), 7.85 (s, 1H), 7.65 (d, 2H, J=8.5 Hz), 7.60 (s, 1H), 7.56 (s, 1H), 7.30 (s, 1H), 6.87 (s, 1H), 5.13 (s, 2H), 3.52-3.69 (m, 15H), 3.39 (q, 2H, J=6.0 Hz), 3.51 (s, 3H), 2.70 (m, 2H), 2.59 (m, 2H), 1.90 (quint, 2H, J=6.1 Hz), 1.74 (quint, 2H, J=5.9 Hz), 1.48 (s, 9H), 1.36 (s, 9H). ¹³C NMR (500 MHz, DMSO-d₆) δ: 171.8, 171.4, 166.2, 165.0, 163.1, 162.2, 159.9, 154.3, 144.2, 141.7, 138.7, 137.0, 134.2, 129.0, 126.0, 119.6, 116.4, 84.4, 78.4, 70.3, 70.0, 68.6, 68.5, 37.2, 36.3, 32.3, 30.5, 29.8, 29.7, 28.4, 27.8. ESI-MS m z calculated for C₄₂H₆₀N₈O₁₃SI (M±H)^{±}: 1043.94; found: 1044.0.

Synthesis of iso-GMIB-PODS (XXI)

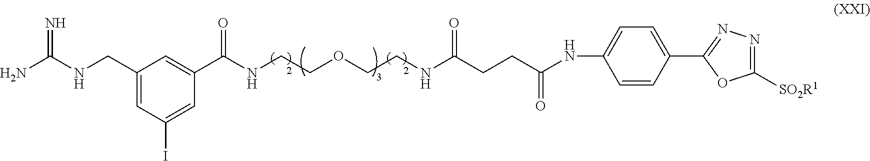

Trifluoroacetic acid (TFA; 0.7 mL) was added to a solution of Boc₂-iso-GMIB-PODS (5.0 mg, 4.8 μmol) in 2.0 mL dichloromethane and the mixture stirred at room temperature for 3 h. The volatiles were evaporated using a rotary evaporator, the crude product re-constituted with 2 mL acetonitrile, and the resultant solution filtered with a syringe filter. The product was purified via RP-HPLC using a preparative column (6 mL/min, 5-95% acetonitrile in water with 0.1% TFA over 35 min; t_{R} of product: 19.3 min). The purity of the isolated sample was assessed using analytical RP-HPLC (1 mL/min, 5-95% acetonitrile in water with 0.1% TFA over 35 min; t_{R} of product: 15.4 min; >95% purity). The pooled HPLC fractions containing the product were lyophilized overnight to yield a white powder (4.0 mg, 87.1% yield; trifluoroacetate salt): ¹H NMR (500 MHz, DMSO-d₆) δ: 10.42 (s, 1H), 8.57 (t, 1H, J=5.3 Hz), 8.12 (s, 1H), 8.03 (m, 3H), 7.86 (m, 3H), 7.79 (d, 2H, J=7.8 Hz), 7.28 (s, 3H), 4.38 (d, 2H, J=6.0 Hz), 3.69 (s, 3H), 3.25-3.54 (m, 14H), 3.07 (q, 2H, J=6.3 Hz), 2.61 (t, 2H, J=6.9 Hz), 2.41 (t, 2H, J=7.0 Hz), 1.74 (quint, 2H, J=6.6 Hz), 1.61 (quint, 2H, J=6.6 Hz). ¹³C NMR (500 MHz, DMSO-d₆) δ: 171.8, 171.5, 166.2, 162.2, 144.1, 129.0, 119.6, 116.4, 70.2, 70.1, 70.0, 68.7, 68.5, 43.4, 40.9, 36.3, 32.2, 30.5, 29.8, 29.1. ESI-MS m z calculated for C₃₂H₄₆N₈O₉SI (M±H)^{±}: 844.7; found: 843.6.

Synthesis of iso-[¹³¹I]GMIB-PODS-5F7GGC (XXII)

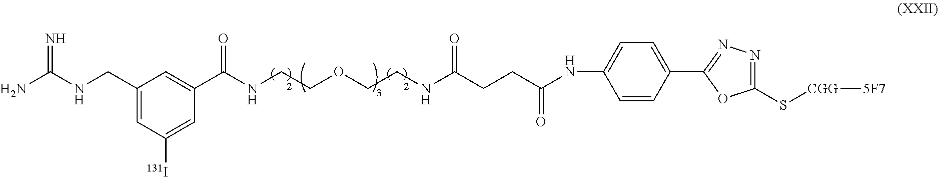

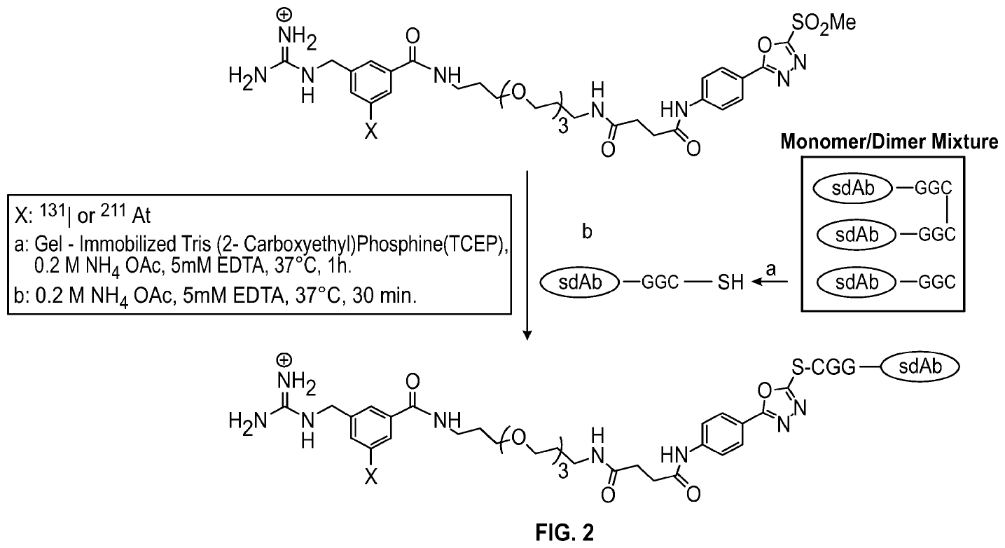

Conjugation of iso-[¹³¹I]GMIB-PODS to monomeric 5F7GGC was performed by adapting a method previously reported for similar compounds (Feng et al. “Site-specific radioiodination of an anti-HER2 single domain antibody fragment with a residualizing prosthetic agent” Nucl. Med. Biol. 2021; 92:171-83). The general synthetic scheme for the synthesis is depicted in FIG. 2. Briefly, a methanolic solution (100 μL) of N-chlorosuccinimide (NCS) (0.2 mg/mL), acetic acid (1%; v/v) and sodium [¹³¹I]iodide (1-5 μL, 37-185 MBq) was added to a half-dram glass vial containing Boc₂-iso-GMTB-PODS (50 μg, 0.05 μmol). The vial was vortexed and the reaction was allowed to proceed at 20° C. for 15 min. The volatiles were evaporated with a stream of argon and the residual activity reconstituted in 40% acetonitrile in water (100 μL). This solution was injected onto a reversed-phase HPLC column eluted at a flow rate of 2 mL/min with a gradient consisting of 0.1% TFA in water (solvent A) and 0.1% TFA in acetonitrile (solvent B); the proportion of B was linearly increased from 40% to 80% over 8 min. The HPLC fractions containing the product (t_{R}=3.0 min) were pooled and most of the acetonitrile was removed using a stream of argon. The product, Boc₂-iso-[¹³¹I]GMIB-PODS, was extracted with 0.5 mL of ethyl acetate and transferred to an half-dram glass vial, and the ethyl acetate was evaporated with argon, TFA (100 μL) added and the deprotection of Boc₂-iso-[¹³¹I]GMIB-PODS was performed at 20° C. for 10 min. Subsequently, TFA was removed with a stream of argon, followed by co-evaporation with ethyl acetate (100 μL×3). Conjugation of iso-[¹³¹I]GMIB-PODS to monomeric 5F7GGC was performed similarly as reported for [¹³¹I]MEGMIB. Briefly, monomeric sdAb 5F7GGC (˜100 μg, 60 μL), freshly obtained as described above, was added to the vial containing iso-[¹³¹I]GMIB-PODS and the conjugation was carried out at 37° C. for 45 min. The labeled sdAb was isolated by gel filtration over a PD-10 column using PBS as the mobile phase. Fractions containing the iso-[¹³¹I]GMIB-PODS-5F7GGC were pooled for use in the biological experiments described below.

Synthesis of iso-[²¹¹At]AGMB-PODS-5F7GGC (XXIII)

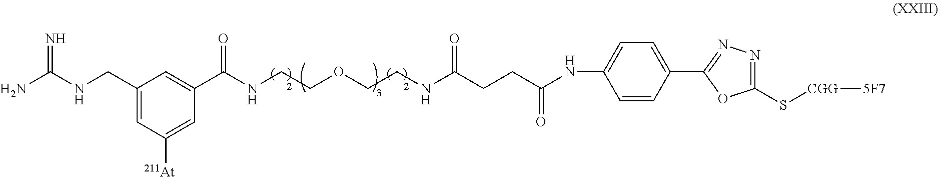

A method previously reported for the synthesis of iso-[²¹¹At]SAGMB (6) was adapted for the synthesis of iso-[²¹¹At]AGMB-PODS at relatively high ²¹¹At activity levels. The general synthetic scheme for the synthesis is depicted in FIG. 2. Astatine-211 in NCS/methanol (˜370 MBq; 200 μL) was added to a vial containing Boc₂-iso-GMTB-PODS (50 μg, 0.05 μmol), the vial vortexed briefly, and the reaction was allowed to proceed at 20° C. for 15 min. The volatiles were evaporated under a stream of argon and the residual activity reconstituted in 40% acetonitrile in water (100 μL). This solution was injected onto a reversed-phase analytical HPLC column eluted with the same gradient described above. The HPLC fractions containing the product, Boc₂-iso-[²¹¹At]AGMB-PODS (t_{R}=3.3 min), were pooled and most of the acetonitrile was removed using a stream of argon. Ethyl acetate (1 mL) was added to extract the Boc₂-iso-[²¹¹At]AGMB-PODS, the mixture was vortexed for 10 s and the ethyl acetate layer was transferred to a half-dram vial. The ethyl acetate was removed using a stream of argon and TFA was added and incubated at room temperature for 10 min. The TFA was evaporated under a stream of argon, and the remaining TFA was removed by co-evaporation with ethyl acetate (100 uL×3). Freshly reduced 5F7GGC was added and the mixture was incubated at 37° C. for 45 min. The iso-[²¹¹At]AGMB-PODS-5F7GGC was isolated using a PD-10 column as described above.

### Synthesis of [²¹¹At]MEAGMB-5F7GGC (XXIV)

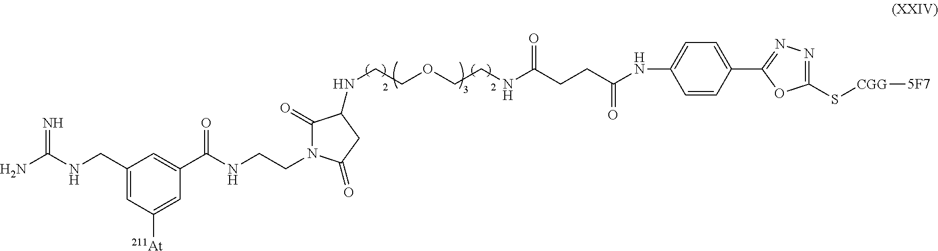

Synthesis of [²¹¹At]MEAGMB and its subsequent conjugation to 5F7GGC was performed by adapting a previously reported method (Choi et al. “Astatine-211 labeled anti-HER2 5F7 single domain antibody fragment conjugates: radiolabeling and preliminary evaluation” Nucl. Med. Biol. 2018; 56:10-20; Feng et al. “Site-specific radioiodination of an anti-HER2 single domain antibody fragment with a residualizing prosthetic agent” Nucl. Med. Biol. 2021; 92:171-83). Briefly, ²¹′At, produced as described previously (Choi et al. “Astatine-211 labeled anti-HER2 5F7 single domain antibody fragment conjugates: radiolabeling and preliminary evaluation” Nucl. Med. Biol. 2018; 56:10-20), in NCS/methanol (˜370 MBq; 200 μL) was added to a vial containing Boc₂-MEGMTB (50 μg, 0.07 μmol), and then 2 μL of glacial acetic acid was added. The vial was vortexed for a short time and the reaction was allowed to proceed at 20° C. for 20 min. The volatiles were evaporated under a stream of argon and the residual activity reconstituted in 40% acetonitrile in water (100 μL). This solution was injected onto a reversed-phase HPLC column eluted a flow rate of 2 mL/min with a gradient consisting of 0.1% TFA in water (solvent A) and 0.1% TFA in acetonitrile (solvent B); the proportion of B was linearly increased from 40% to 80% over 8 min. The HPLC fractions containing the product (t_{R}=4.0 min) were pooled and most of the acetonitrile was removed using a stream of argon. Boc₂-[²¹¹At]MEAGMB was extracted with 2×1 mL of ethyl acetate and transferred to a half-dram glass vial. Ethyl acetate was evaporated with argon, TFA (100 μL) was added and the deprotection of Boc₂-[²¹¹At]MEAGMB was allowed to proceed at 20° C. for 10 min. Subsequently, TFA was removed under a stream of argon, followed by co-evaporation with ethyl acetate (100 μL×3). Monomeric sdAb 5F7GGC (˜100 μg, 60 μL), freshly obtained as described above, was added to the vial containing [²¹¹At]MEAGMB and the conjugation was carried out at 37° C. for 45 min. The labeled sdAb was isolated by gel filtration over a PD-10 column using PBS as the mobile phase. Fractions containing [²¹¹At]MEAGMB-5F7GGC were pooled for use in the biological experiments described below.

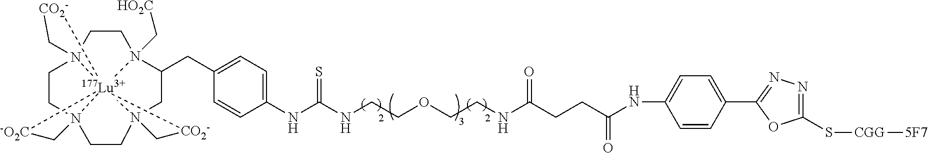

### Synthesis of [¹⁷⁷Lu]Lu-DOTA-PODS-5F7GGC

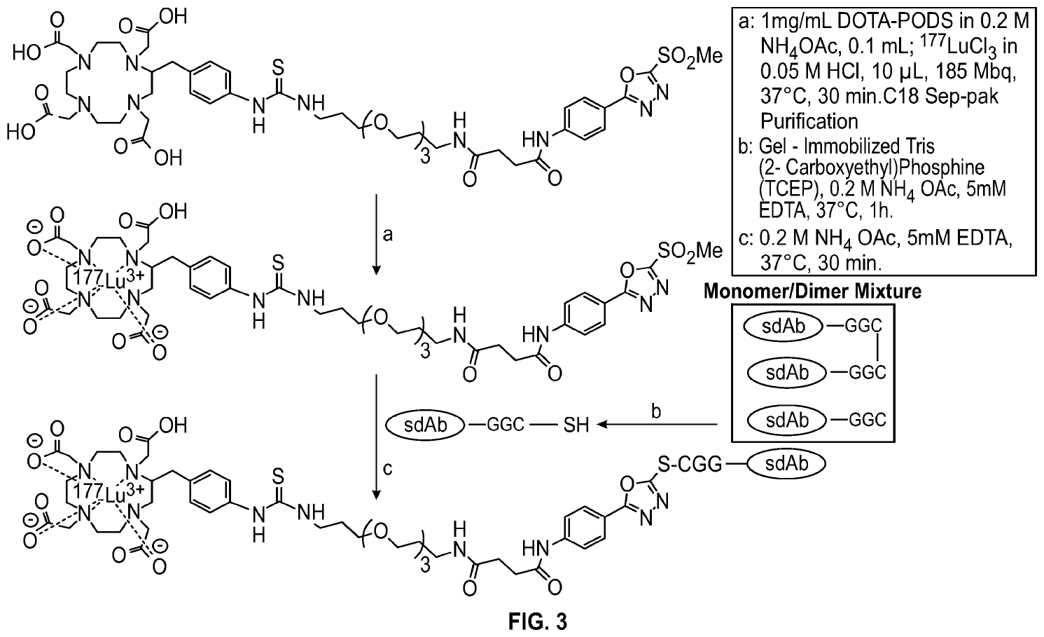

The general synthetic scheme for the synthesis of [¹⁷⁷Lu]Lu-DOTA-PODS-5F7GGC is shown in FIG. 3. Lutetium-177, produced at the University of Missouri Research Reactor, was purchased from the National Isotope Development Center, Oak Ridge National Laboratory. Upon receipt, ¹⁷⁷Lu (185 MBq, 10 μL) was diluted with 0.1 M HCl (50 μL) and added to a 1 mL Eppendorf tube and DOTA-PODS (150 μL, 1 mg/mL, in 0.2 M NH₄OAc, pH=6.3) was added. The reaction mixture was vortexed for 30 s and placed at room temperature for 30 min. After that, the reaction mixture was diluted with 10 mL water and passed through a preconditioned C18 Sep-Pak® cartridge (Waters; 60 mg). The product activity was eluted from the cartridge with ethanol (400 μL) into a half-dram glass vial and the ethanol was evaporated with a gentle stream of argon. Monomeric 5F7GGC sdAb, freshly obtained as described above, was added to the vial containing [¹⁷⁷Lu]Lu-DOTA-PODS (180 MBq) and the conjugation was carried out at 37° C. for 45 min. After that, the conjugate was treated with 50 mM EDTA to remove any adventitiously bound ¹⁷⁷Lu. The labeled sdAb was isolated by gel filtration over a PD-10 column using PBS as the mobile phase. Fractions containing the [¹⁷⁷Lu]Lu-DOTA-PODS-5F7GGC were pooled for use in the biological experiments described below.

### Cell Culture Conditions

Reagents for cell culture were obtained from Thermo Fisher Scientific (Waltham, MA), except where noted. Cells were cultured at 37° C. in a 5% C02 humidified incubator. HER2-positive BT474 human breast carcinoma cells were obtained from Duke University Cell Culture Facility and grown in Dulbecco's Modified Eagle's Medium (DMEM) containing 10% FBS, supplemented with 10 mg/mL bovine insulin. SKOV-3 human ovarian carcinoma cells were obtained from the Duke University Cell Culture Facility and grown in McCoy's 5A medium containing 10% fetal bovine serum and 1% penicillin-streptomycin.

Quality Control of Labeled sdAb Conjugates

The radiochemical purity of labeled sdAb was evaluated by SDS-PAGE/phosphor imaging. The immunoreactive fraction was determined by the Lindmo method (Lindmo, T., et al., “Determination of the immunoreactive function of radiolabeled monoclonal antibodies by linear extrapolation to binding at infinite antigen excess.” Journal of Immunological Methods, 1984. 72(1): p. 77-89, incorporated herein by reference) following a reported procedure (Foulon, C. F., et al., “Radioiodination via D-amino acid peptide enhances cellular retention and tumor xenograft targeting of an internalizing anti-epidermal growth factor receptor variant III monoclonal antibody.” Cancer Research, 2000. 60(16): p. 4453-4460, incorporated herein by reference). HER2 binding affinity of iso-[¹³¹I]GMIB-PODS-5F7GGC was determined using HER2-expressing SKOV-3 and BT474 cells by the saturation binding assay as described before (Zhou, Z., et al., “Fluorine-18 labeling of an anti-HER2 VHH using a residualizing prosthetic group via a strain-promoted click reaction: Chemistry and preliminary evaluation.” Bioorganic & Medicinal Chemistry, 2018. 26(8): p. 1939-1949 and Vaidyanathan, G., et al., “N-Succinimidyl 3-((4-(4-[¹⁸F] fluorobutyl)-1 H-1, 2, 3-triazol-1-yl) methyl)-5-(guanidinomethyl) benzoate ([¹⁸F] SFBTMGMB): a residualizing label for ¹⁸F-labeling of internalizing biomolecules.” Organic & Biomolecular Chemistry, 2016. 14(4): p. 1261-1271, both of which are incorporated herein by reference. Nonspecific binding was determined in parallel assays by co-incubating cells with 100-fold molar excess of trastuzumab.

### Determination of Internalization In Vitro

Internalization and cellular retention of iso-[¹³¹I]GMIB-PODS-5F7GGC in BT474 breast carcinoma cells was assessed using procedures reported for similar molecules (Choi, J., et al., Astatine-211 labeled anti-HER2 5F7 single domain antibody fragment conjugates: Radiolabeling and preliminary evaluation. Nuclear Medicine and Biology, 2018. 56: p. 10-20 and Zhou, Z., et al., Fluorine-18 labeling of the HER2-targeting single-domain antibody 2Rs15d using a residualizing label and preclinical evaluation. Molecular Imaging and Biology, 2017. 19(6): p. 867-877, both of which are incorporated herein by reference. Briefly, cells were incubated with the radiolabeled sdAb and the internalized fractions were measured at different time points. A paired-label assay of iso-[¹³¹I]GMIB-PODS-5F7GGC in tandem with [¹²⁵I]MEGMIB-5F7GGC was performed in BT474 cells following reported procedures (Feng, Y., et al., Site-specific radioiodination of an anti-HER2 single domain antibody fragment with a residualizing prosthetic agent. Nuclear Medicine and Biology, 2021. 92: p. 171-183. and Choi, J., et al., Astatine-211 labeled anti-HER2 5F7 single domain antibody fragment conjugates: Radiolabeling and preliminary evaluation. Nuclear Medicine and Biology, 2018. 56: p. 10-20.) Briefly, cells were incubated with the radiolabeled sdAbs at 4° C. for 30 min, then washed with PBS and replaced with fresh medium, and the internalized fractions were measured at different time points. In these assays, nonspecific uptake was assessed in parallel experiments by co-incubating cells with 100-fold molar excess of trastuzumab.

In vitro Stability of[²¹¹At]MEAGMB-5F7GGC and iso-[²¹¹At]AGMB-PODS-5F7GGC

The in vitro stability of the ²¹¹At-labeled conjugates was evaluated in PBS, 50 mM cysteine solution and human serum albumin (50% in PBS). Briefly, the ²¹¹At-labeled sdAb conjugate (3.7 MBq, ˜12 μg sdAb) was added to 1 mL of each solution in 10 mL centrifuge tubes. The tubes containing the conjugates were vortexed for 10 s and left at room temperature. The radiochemical purity of each conjugate was determined by SDS-PAGE and phosphor imaging of aliquots of the incubates after 3 h and 21 h.

### Biodistribution

All experiments involving animals were performed under a protocol approved by the Duke University IACUC. Subcutaneous BT474 breast carcinoma xenografts were established by inoculating 5-week old female athymic mice (˜25 g) with 20×10⁶ BT474 cells in 50% protein/peptide comprises a C-terminal GGC tail and wherein the radiolabel is coupled to the protein/peptide through the C-terminal GGC tail Matrigel (Corning Inc., NY) in the above medium (100 μL), after implanting estrogen pellet (17β-Estradiol) subcutaneously in the back of the neck. Four biodistribution studies were performed: iso-[¹³¹I]GMIB-PODS-5F7GGC in tandem with [¹²⁵I]MEGMIB-5F7GGC in paired-label fashion in athymic mice without xenografts; iso-[¹³¹I]GMIB-PODS-5F7GGC in tandem with iso-[²¹¹At]AGMB-PODS-5F7GGC in paired-label fashion in athymic mice bearing BT474 xenografts; iso-[¹³¹I]GMIB-PODS-5F7GGC in tandem with [¹²⁵I]MEGMIB-5F7GGC in paired-label fashion in athymic mice bearing BT474 xenografts. In these studies, each mouse received 0.11-0.22 MBq (1-2 μg of sdAb) of each labeled conjugate via the tail vein. At each time point after the tracer administration, blood and urine were collected and the mice were killed by an overdose of isofluorane. Tumor and other tissues were harvested, blot-dried, weighed and counted along with the relevant injection standards for ¹²⁵I, ¹³¹I and ²¹¹At activity using an automated gamma counter. From these counts, the percentage of the injected dose (% ID) per organ, per gram of tissue (% ID/g) and tumor-to-normal tissue ratios were calculated.

### Chemistry and Radiochemistry Results

The syntheses of Boc₂-iso-SGMIB, Boc₂-iso-SGMTB and PODS have been reported before (Feng, Y., et al., Site-specific radioiodination ofan anti-HER2 single domain antibody fragment with a residualizing prosthetic agent. Nuclear Medicine and Biology, 2021. 92: p. 171-183; Adumeau, P., M. Davydova, and B. M. Zeglis, Thiol-reactive bifunctional chelators for the creation of site-selectively modified radioimmunoconjugates with improved stability. Bioconjugate Chemistry, 2018. 29(4): p. 1364-1372; and Davydova, M., et al., Synthesis and bioconjugation of thiol-reactive reagents for the creation of site-selectively modified immunoconjugates. Journal of Visualized Experiments: JoVE, 2019(145)). The conjugation of Boc₂-iso-SGMIB and Boc₂-iso-SGMTB to PODS, respectively, were performed using a common protocol under basic conditions. The product Boc₂-iso-GMIB-PODS and Boc₂-iso-GMTB-PODS were separated using preparative reverse phase HPLC in reasonable yields. Iso-GMIB-PODS-5F7GGC, DOTA-PODS-5F7GGC, and Lu-DOTA-PODS-5F7GGC were synthesized in similar yield (˜76%). The purities of these immunoconjugates were determined using GP HPLC and all were >95%. Their molecular weights were determined via LC-MS and were 13795.4 (13795.2 calc.), 14046.0 (14046.2 calc.), and 14216.7 (14217.2 calc.), respectively. Non-radioactive iso-SGMIB-PODS was used as a standard for the identification of its radiolabeled analogue, iso-[¹³¹I]GMIB-PODS. The radiochemical yield (RCY) and radiochemical purity (RCP) of Boc₂-iso-[¹³¹I]GMIB-PODS were 70±8% (n=11) and >99% (RP-HPLC), respectively. The conjugation of iso-[¹³¹I]GMIB-PODS to monomeric 5F7GGC was accomplished with a RCY of 58±9% (n=11); the RCP of iso-[¹³¹I]GMIB-PODS-5F7GGC determined by SDS-PAGE and GP-HPLC was >99%. The synthesis of iso-[²¹¹At]AGMB-PODS was performed with a RCY of 66±5% (n=6), and the RCP was >99% for each synthesis. The RCY for the conjugation of iso-[²¹¹At]AGMB-PODS with 5F7GGC was 64±7% (n=6), and the RCP of the radiolabeled sdAb determined by SDS-PAGE and GP-HPLC was >99%. [¹⁷⁷Lu]Lu-DOTA-PODS was synthesized in almost quantitative yields, and [¹⁷⁷Lu]Lu-DOTA-PODS-5F7GGC was synthesized in 35±15% RCY (n=2) and >99% RCP as determined by SDS-PAGE and GP-HPLC.

Characterization of sdAb Conjugates

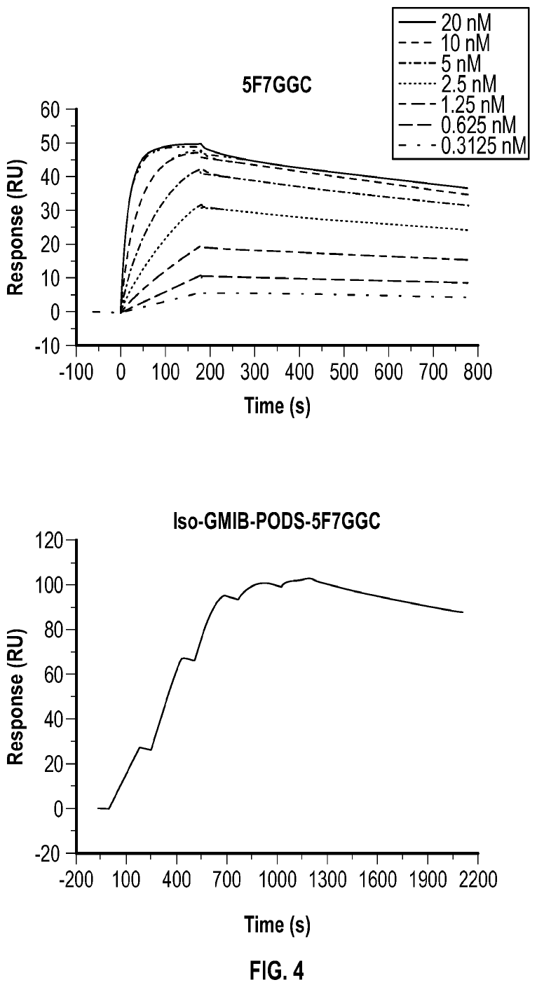

The binding affinities (Kd) of 5F7GGC and 5F7GGC conjugates for HER2 extracellular domain measured by surface plasmon resonance (SPR). The Kd for 5F7GGC was determined using multi-cycle kinetic titration and 5F7GGC conjugates were determined using single-cycle kinetic titration. Results are shown in FIG. 4 and Table 1. SPR revealed binding constants (K_{d}) of 0.10, 0.19, and 0.09 nM for iso-GMIB-PODS-5F7GGC, DOTA-PODS-5F7GGC and Lu-DOTA-PODS-5F7GGC, respectively, with recombinant HER2-Fc protein. The immunoreactive fraction (IRF), determined by Lindmo assay (Lindmo “Determination of the immunoreactive function of radiolabeled monoclonal antibodies by linear extrapolation to binding at infinite antigen excess.” J Immunol. Methods. 1984; 72(1):77-89.), was 84.0±1.8% (n=2) for iso-[¹³¹I]GMIB-PODS-5F7GGC, 85.8±2.1% (n=2) for iso-[²¹¹At]AGMB-PODS-5F7GGC, 69.0% (n=1) for [²¹¹At]MEAGMB-5F7GGC, and 71.2% (n=1) for [¹⁷⁷Lu]Lu-DOTA-PODS-5F7GGC.

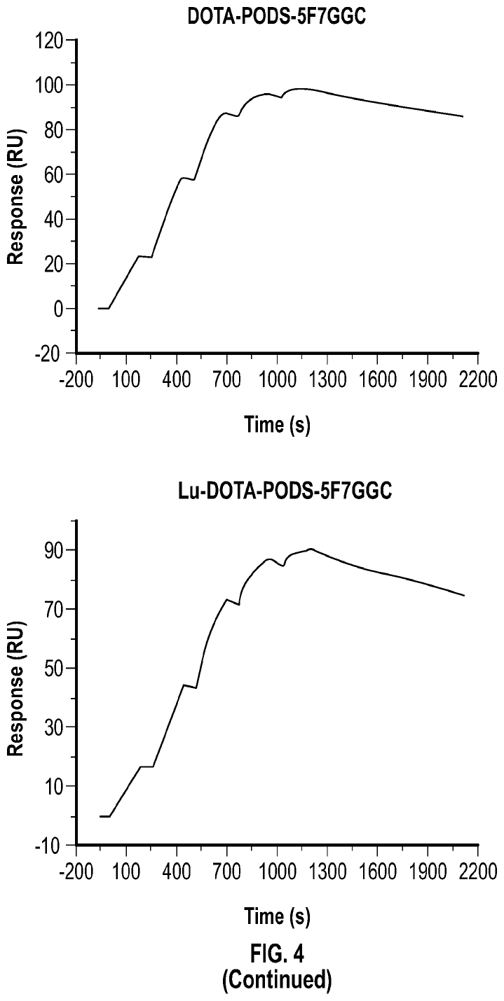

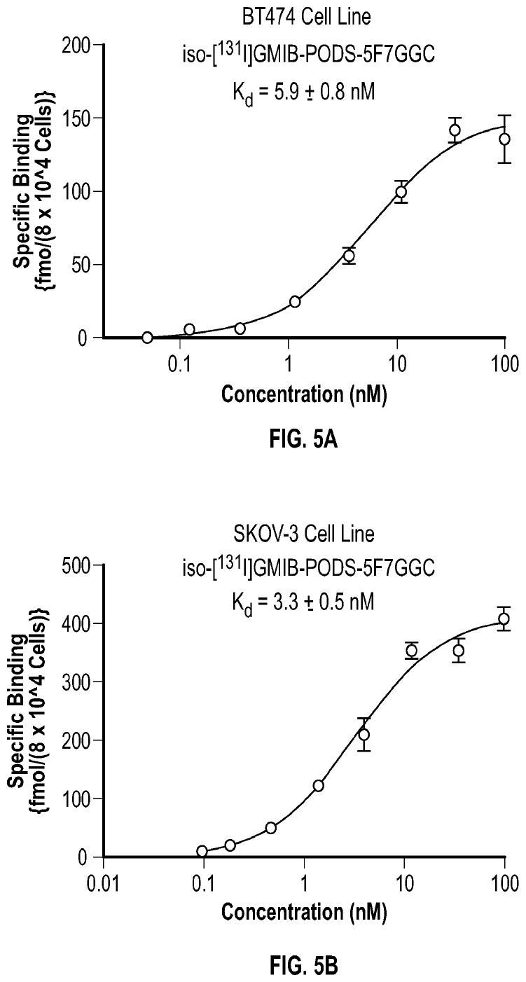

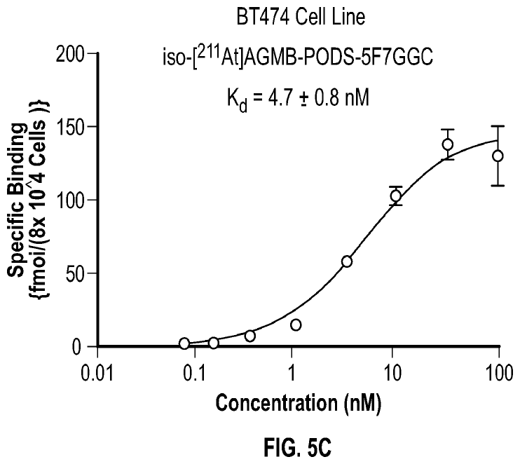

FIG. 5A shows the binding affinity of iso-[¹³¹I]GMIB-PODS-5F7GGC measured in BT474 cells. FIG. 5B shows binding affinity of iso-[¹³¹I]GMIB-PODS-5F7GGC measured in SKOV-3 cells. FIG. 5C shows binding affinity of iso-[²¹¹At]AGMB-PODS-5F7GGC measured in BT474 cells. As shown in the figures, saturation binding assays using the HER2-expressing SKOV-3 and BT474 cell lines gave K_{d} values of 3.3±0.5 nM and 5.9±0.8 nM, respectively, for iso-[¹³¹I]GMIB-PODS-5F7GGC. Using the BT474 cell line, K_{d} values of 4.7±0.8, 3.4±0.6, and 5.6±0.9 nM were obtained for iso-[²¹¹At]AGMB-PODS-5F7GGC, [²¹¹At]MEAGMB-5F7GGC, and [¹⁷⁷Lu]Lu-DOTA-PODS-5F7GGC, respectively.

TABLE 1

| | | [nM] | | | | | |
|---|---|---|---|---|---|---|---|
| | | range | | | | | |
| Ligand | Sample | studied | Model | kₐ (1/Ms) | k_{d} (1/s) | K_{d} (M) | K_{d} (nM) |
| Her2Fc | 5F7GGC | 1.64-25 | 1:1 | 2.94E+06 | 4.80E−04 | 1.64E−10 | 0.16 |
| Her2Fc | isOGMIB-PODS- | 1.56-25 | 1:1 | 1.45E+06 | 1.42E−04 | 9.79E−11 | 0.10 |
| | 5F7GGC | | | | | | |
| Her2Fc | DOTA-PODS- | 1.56-25 | 1:1 | 1.13E+06 | 2.12E−04 | 1.88E−10 | 0.19 |
| | 5F7GGC | | | | | | |
| Her2Fc | Lu-DOTA-PODS- | 1.56-25 | 1:1 | 1.96E+06 | 1.68E−04 | 8.57E−11 | 0.09 |
| | 5F7GGC | | | | | | |

### Cellular Retention and Internalization

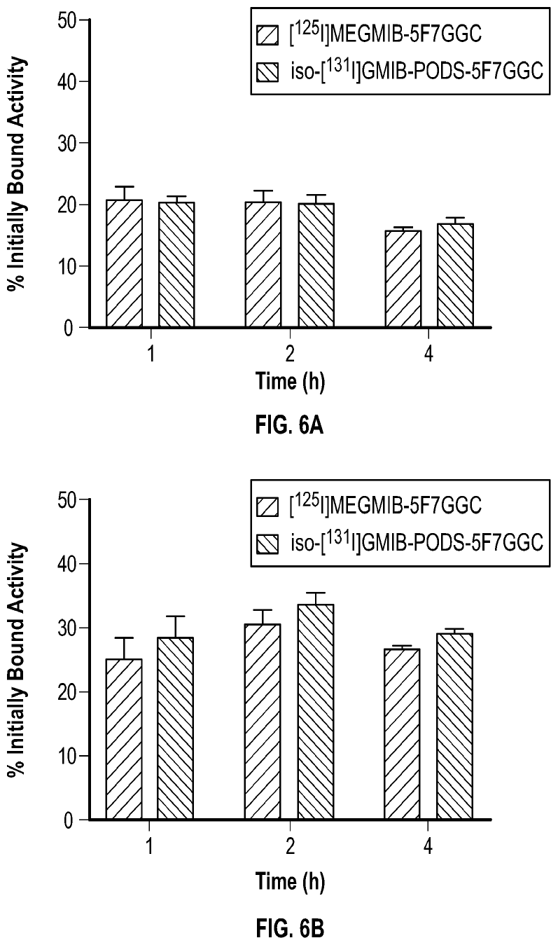

FIG. 6 shows in vitro paired-label internalization assay on HER2-positive BT474 breast cancer carcinoma cells co-incubated with iso-[¹²⁵I]MEGMIB-5F7GGC and iso-[¹³¹I]GMIB-PODS-5F7GGC. The results are presented as surface-bound (FIG. 6A) and internalized (FIG. 6B) fraction of the radioactivity initially bound to the cells after a 1 h incubation at 4° C. In the paired-label internalization assay comparing iso-[¹³¹I]GMIB-PODS-5F7GGC and [¹²⁵I]MEGMIB-5F7GGC, 6.2±0.3% of iso-[¹³¹I]GMIB-PODS-5F7GGC was associated with cells after the initial 1 h incubation at 4° C. compared with 5.8±0.4% for [¹²⁵I]MEGMIB-5F7GGC (difference not statistically significant: P>0.05). The fraction of this initially bound iso-[¹³¹I]GMIB-PODS-5F7GGC that was internalized in the cells after subsequent incubation at 37° C. was 28.4±3.3%, 33.5±1.8%, and 29.0±0.8% at 1, 2, and 4 h, respectively. For [¹²⁵I]MEGMIB-5F7GGC, these values were 25.0±3.4%, 30.5±2.2%, and 26.7±0.5%. A significantly higher internalized fraction was only observed for iso-[¹³¹I]GMIB-PODS-5F7GGC compared to [¹²⁵I]MEGMIB-5F7GGC at 4 h (P<0.05). The surface-bound fractions for iso-[¹³¹I]GMIB-PODS-5F7GGC and [¹²⁵I]MEGMIB-5F7GGC were not significantly different at any time point.

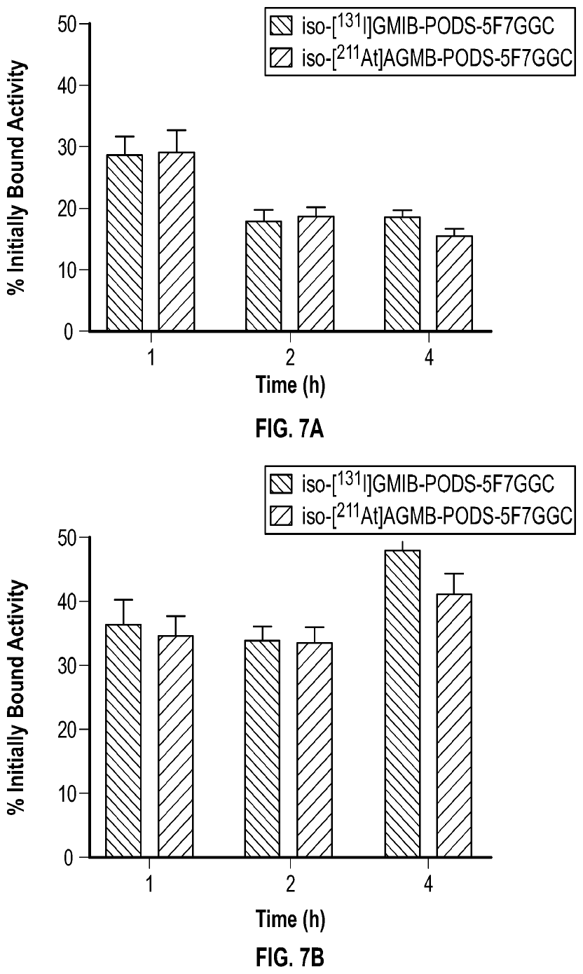

FIG. 7 shows in vitro paired-label internalization assay performed on HER2-positive BT474 breast carcinoma cells co-incubated with iso-[¹³¹I]GMIB-PODS-5F7GGC and iso-[²¹¹At]AGMB-PODS-5F7GGC. The results are presented as surface-bound (FIG. 7A) and internalized (FIG. 7B) fraction of the radioactivity initially bound to the cells after a 1 h incubation at 4° C. The cellular uptake and internalization of iso-[¹³¹I]GMIB-PODS-5F7GG were compared with that of iso-[²¹¹At]AGMB-PODS-5F7GGC in the paired-label assay. No significant difference was observed in the uptake after 1 h of incubation at 4° C., with 7.4±0.8% and 7.5±1.0% of input activity bound to BT474 cells for iso-[¹³¹I]GMIB-PODS-5F7GGC and [²¹¹At]AGMB-PODS-5F7GGC, respectively. The surface-bound fractions for iso-[¹³¹I]GMIB-PODS-5F7GGC ranged from 17.7% to 28.5% of the initially bound activity, and the internalized fractions were between 34.4% and 48.8%. For iso-[²¹¹At]AGMB-PODS-5F7GGC, the surface bound fractions were between 15.4% and 29.0%, and the internalized fractions were between 34.1% and 41.8%. The differences between the ¹³¹I—and ²¹¹At-labeled sdAbs were not statistically significant at any time point.

In vitro Stability of [²¹¹At]MEAGMB-5F7GGC and iso-[²¹¹At]AGMB-PODS-5F7GGC

Iso-[²¹¹At]AGMB-PODS-5F7GGC showed excellent stability in both PBS and a cysteine solution, with over 95% RCP after 21 h. A decrease in stability was observed in HSA, with 86% and 76% of the ²¹¹At remaining associated with the sdAb at 3 and 21 h, respectively. Compared with iso-[²¹¹At]AGMB-PODS-5F7GGC, the in vitro stability of [²¹¹At]MEAGMB-5F7GGC in cysteine solution and HSA was considerably lower, while no significant difference was observed between the two ²¹¹At-labeled sdAbs in PBS. Notably, virtually no radioactivity was associated with intact sdAb at 3 and 21 h for [²¹¹At]MEAGMB-5F7GGC in both the cysteine solution and HSA.

### Biodistribution

The paired-label biodistribution of iso-[¹³¹I]GMIB-PODS-5F7GGC and [¹²⁵I]MEGMIB-5F7GGC were compared in athymic mice both without (Table 2) and with BT474 subcutaneous xenografts (Table 3). In the mice without tumors, the uptake of radioactivity in the kidneys from iso-[¹³¹I]GMIB-PODS-5F7GGC at 1 h post-injection (p.i.) (36.3±5.2% ID/g) was significantly higher than that from [¹²⁵I]MEGMIB-5F7GGC (15.7±1.3% ID/g; P<0.005). However, at 4 h p.i., the kidney activity concentrations produced by both radioimmunoconjugates were not significantly different (P>0.05). The uptake of iso-[¹³¹I]GMIB-PODS-5F7GGC in the liver at 1 h p.i. was significantly higher (P<0.01) than that from [¹²⁵I]MEGMIB-5F7GGC with the trend reversed at 4 h p.i., (P<0.05). Minimal uptake and retention was observed in other organs. As shown in Table 3, the uptake of iso-[¹³¹I]GMIB-PODS-5F7GGC in the BT474 tumors was not significantly different (P>0.05 for all time points) from that for [¹²⁵I]MEGMIB-5F7GGC. The differences in the tissue concentrations in normal organs were consistent with those observed in mice without tumors. At 1 h p.i., the tumor-to-kidney activity concentration ratio for [¹²⁵I]MEGMIB-5F7GGC (0.8±0.1) was significantly higher than that for iso-[¹³¹I]GMIB-PODS-5F7GGC (0.3±0.1; P<0.0001); however, the tumor-to-kidney activity concentration ratios of the two conjugates were not significantly different at 4 h p.i. The tumor-to-liver activity concentration ratios for [¹²⁵I]MEGMIB-5F7GGC were 9.2±1.5, 23.3±6.8, and 63.4±21.2 at 1, 4 and 24 h p.i., respectively, values significantly higher (P<0.0001) than those for iso-[¹³¹I]GMIB-PODS-5F7GGC at each of the time points.

TABLE 2
[¹²⁵I]MEGMIB-5F7GGC/[¹³¹I]GMIB-PODS-5F7GGC
(No Tumor)
Percent injected dose per gram^{a}

| | 1 h | | 4 h | | 24 h | |
|---|---|---|---|---|---|---|
| Tissue | ¹²⁵I | ¹³¹I | ¹²⁵I | ¹³¹I | ¹²⁵I | ¹³¹I |
| Liver | 3.1 ± 0.4 | 5.2 ± 0.6 | 1.2 ± 0.2 | 0.7 ± 0.3 | 0.4 ± 0.1 | 0.1 ± 0.0 |
| Spleen | 1.4 ± 0.6 | 1.2 ± 0.5 | 0.7 ± 0.1 | 0.4 ± 0.1 | 0.3 ± 0.1 | 0.1 ± 0.0 |
| Lungs | 2.4 ± 0.4 | 3.4 ± 1.2 | 1.2 ± 0.1 | 1.2 ± 0.4 | 0.3 ± 0.2 | 0.4 ± 0.3 |
| Heart | 0.4 ± 0.0 | 0.5 ± 0.1 | 0.1 ± 0.0 | 0.0 ± 0.0 | 0.0 ± 0.0 | 0.0 ± 0.0 |
| Kidneys | 15.7 ± 1.3 | 36.3 ± 5.2 | 1.6 ± 0.2 | 1.9 ± 0.4 | 0.6 ± 0.1 | 0.2 ± 0.0 |
| Stomach | 0.6 ± 0.1 | 0.8 ± 0.3 | 0.3 ± 0.1 | 0.3 ± 0.2 | 0.0 ± 0.0 | 0.0 ± 0.0 |
| Sm. Int. | 2.8 ± 0.3 | 4.3 ± 0.7 | 0.8 ± 0.1 | 0.9 ± 0.3 | 0.0 ± 0.0 | 0.0 ± 0.0 |
| Lg. Int. | 0.6 ± 0.1 | 0.5 ± 0.1 | 3.9 ± 0.4 | 6.4 ± 0.7 | 0.1 ± 0.0 | 0.1 ± 0.0 |
| Muscle | 0.3 ± 0.1 | 0.3 ± 0.1 | 0.0 ± 0.0 | 0.0 ± 0.0 | 0.0 ± 0.0 | 0.0 ± 0.0 |
| Blood | 0.7 ± 0.5 | 0.8 ± 0.4 | 0.1 ± 0.0 | 0.1 ± 0.0 | 0.0 ± 0.0 | 0.0 ± 0.0 |
| Bone | 0.4 ± 0.1 | 0.5 ± 0.1 | 0.1 ± 0.0 | 0.1 ± 0.0 | 0.0 ± 0.0 | 0.0 ± 0.0 |
| Brain | 0.0 ± 0.0 | 0.0 ± 0.0 | 0.0 ± 0.0 | 0.0 ± 0.0 | 0.0 ± 0.0 | 0.0 ± 0.0 |
| Thyroid | 0.2 ± 0.1 | 0.2 ± 0.1 | 0.1 ± 0.4 | 0.1 ± 0.1 | 0.0 ± 0.0 | 0.2 ± 0.0 |

^{a}% ID/g, mean ± SD, n = 5

TABLE 3
[¹²⁵I]MEGMIB-5F7GGC/[¹³¹I]GMIB-PODS-5F7GGC
(HER2-positive BT474 xenografts)
Percent injected dose per gram^{a}

| | 1 h | | 4 h | | 24 h | |
|---|---|---|---|---|---|---|
| Tissue | ¹²⁵I | ¹³¹I | ¹²⁵I | ¹³¹I | ¹²⁵I | ¹³¹I |
| Liver | 6.6 ± 1.9 | 1.5 ± 0.4 | 1.4 ± 0.2 | 0.5 ± 0.1 | 0.2 ± 0.1 | 0.1 ± 0.0 |
| Spleen | 0.6 ± 0.2 | 0.4 ± 0.2 | 0.1 ± 0.0 | 0.1 ± 0.0 | 0.0 ± 0.0 | 0.0 ± 0.0 |
| Lungs | 2.4 ± 1.2 | 1.4 ± 0.3 | 1.0 ± 0.3 | 0.7 ± 0.2 | 0.1 ± 0.1 | 0.2 ± 0.1 |
| Heart | 0.5 ± 0.1 | 0.5 ± 0.2 | 0.1 ± 0.0 | 0.1 ± 0.0 | 0.0 ± 0.0 | 0.1 ± 0.0 |
| Kidneys | 55.3 ± 14.1 | 16.2 ± 3.3 | 4.8 ± 0.3 | 2.9 ± 0.4 | 0.7 ± 0.2 | 1.2 ± 0.3 |
| Stomach | 0.8 ± 0.1 | 0.7 ± 0.2 | 1.8 ± 3.5 | 1.5 ± 3.0 | 0.1 ± 0.1 | 0.1 ± 0.1 |
| Sm. Int. | 5.6 ± 0.1 | 9.1 ± 1.7 | 3.1 ± 0.7 | 1.6 ± 0.7 | 0.1 ± 0.1 | 0.1 ± 0.0 |
| Lg. Int. | 0.4 ± 0.1 | 1.1 ± 0.4 | 12.8 ± 3.6 | 14.3 ± 2.6 | 0.4 ± 0.2 | 0.2 ± 0.1 |
| Muscle | 0.5 ± 0.4 | 0.7 ± 0.6 | 0.1 ± 0.0 | 0.1 ± 0.0 | 0.0 ± 0.0 | 0.0 ± 0.0 |
| Blood | 0.8 ± 0.2 | 0.8 ± 0.2 | 0.1 ± 0.0 | 0.3 ± 0.0 | 0.0 ± 0.0 | 0.13 ± 0.0 |
| Bone | 1.2 ± 1.6 | 1.5 ± 1.9 | 0.1 ± 0.0 | 0.1 ± 0.0 | 0.0 ± 0.0 | 0.0 ± 0.0 |
| Brain | 0.1 ± 0.0 | 0.1 ± 0.1 | 0.0 ± 0.0 | 0.0 ± 0.0 | 0.0 ± 0.0 | 0.0 ± 0.0 |
| Thyroid | 0.1 ± 0.4 | 0.0 ± 0.6 | 0.2 ± 0.1 | 0.1 ± 0.1 | 0.2 ± 0.2 | 0.1 ± 0.1 |
| Tumor | 16.2 ± 4.9 | 13.7 ± 4.1 | 13.7 ± 4.1 | 12.1 ± 3.6 | 4.8 ± 1.5 | 5.7 ± 1.8 |

^{a}% ID/g, mean ± SD, n = 5

The biodistribution of iso-[¹³¹I]GMIB-PODS-5F7GGC and its ²¹¹At-labeled analogue iso-[²¹¹At]AGMB-PODS-5F7GGC, were directly compared in a paired-label experiment using athymic mice bearing BT474 xenografts (Table 4). High tumor uptake and retention was observed for both agents, and differences between the two agents were not statistically significant (P>0.05 at 1, 4, and 21 h). Kidney activity levels at 1 h p.i. were also similar; however, at 4 and 21 h p.i., the activity concentration of iso-[²¹¹At]AGMB-PODS-5F7GGC in the kidneys was significantly higher than that for iso-[¹³¹I]GMIB-PODS-5F7GGC (P<0.05 for 4 and 21 h). The tumor-to-kidney activity concentration ratios for iso-[¹³¹I]GMIB-PODS-5F7GGC at 4 and 21 h p.i., respectively, were 2.8±0.5, and 8.0±1.2, significantly higher than those for iso-[²¹¹At]AGMB-PODS-5F7GGC: 1.5±0.3 and 2.7±0.4 at (P<0.01 at 4 h; P<0.0001 at 21 h; no significant difference was seen at 1 h). A similar trend was seen in the liver. At 1 h p.i., no significant difference was seen between the tumor-to-liver activity concentration ratios of the ²¹¹At-labeled (2.5±0.6) and ¹³¹I-labeled analogue (2.8±0.8). However, significantly higher tumor-to-liver activity concentration ratios were seen for the ¹³¹I-labeled sdAb thereafter: 12.2±3.5 and 27.1±8.9 at 4 and 21 h for ¹³¹I, and 7.7±1.8 and 11.6±3.4 for ²¹¹At at 4 and 21 h, respectively, (P<0.05 at 4 h and P<0.01 at 21 h). The activity concentrations in the stomach for iso-[²¹¹At]AGMB-PODS-5F7GGC were significantly higher than those observed for iso-[¹³¹I]GMIB-PODS-5F7GGC at 1 h (P<0.05) and 4 h. Likewise, the uptake of radioactivity in the thyroid for iso-[²¹¹At]AGMB-PODS-5F7GGC was significantly higher than observed for iso-[¹³¹I]GMIB-PODS-5F7GGC at all time points. Nonetheless, the absolute levels of activity in the stomach and thyroid for both radioimmunoconjugates were consistent with a low level of in vivo dehalogenation for both labeled sdAbs but with the ²¹¹At-labeled conjugate showing a higher degree of dehalogenation. When iso-[²¹¹At]AGMB-PODS-5F7GGC was evaluated as a single agent in athymic mice bearing BT474 xenografts, tissue activity levels were consistent with those seen in the paired-label study, except for tumor uptake at 4 h and 24 h (Table 5). In the single-label study, the tumor uptake was two-fold higher: 20.5±2.4% ID/g and 8.8±0.8% ID/g at 4 and 24 h, respectively.

TABLE 4
iso-[¹³¹I] GMIB-PODS-5F7GGC/iso-[²¹¹At]AGMB-PODS-5F7GGC
(HER2-positive BT474 xenografts)
Percent injected dose per gram^{a}

| | 1 h | | 4 h | | 24 h | |
|---|---|---|---|---|---|---|
| Tissue | ¹³¹I | ²¹¹At | ¹³¹I | ²¹¹At | ¹³¹I | ²¹¹At |
| Liver | 4.5 ± 2.2 | 5.0 ± 2.2 | 0.9 ± 0.2 | 1.4 ± 0.2 | 0.2 ± 0.1 | 0.4 ± 0.1 |
| Spleen | 0.4 ± 0.1 | 1.8 ± 0.3 | 0.1 ± 0.0 | 1.3 ± 0.2 | 0.0 ± 0.0 | 0.4 ± 0.2 |
| Lungs | 1.9 ± 0.5 | 4.8 ± 0.2 | 1.4 ± 0.3 | 3.6 ± 0.2 | 0.3 ± 0.2 | 0.8 ± 0.4 |
| Heart | 0.4 ± 0.2 | 0.9 ± 0.3 | 0.0 ± 0.0 | 0.5 ± 0.0 | 0.0 ± 0.0 | 0.1 ± 0.1 |
| Kidneys | 52.5 ± 18.6 | 53.1 ± 17.5 | 4.0 ± 1.0 | 7.0 ± 1.3 | 0.6 ± 0.2 | 1.5 ± 0.4 |
| Stomach | 0.5 ± 0.3 | 3.2 ± 1.2 | 0.1 ± 0.1 | 4.5 ± 0.9 | 0.1 ± 0.1 | 0.9 ± 0.5 |
| Sm. Int. | 3.3 ± 1.1 | 3.8 ± 1.1 | 0.9 ± 0.2 | 1.4 ± 0.3 | 0.2 ± 0.1 | 0.3 ± 0.1 |
| Lg. Int. | 0.3 ± 0.1 | 0.5 ± 0.1 | 6.2 ± 0.5 | 6.4 ± 0.5 | 0.4 ± 0.2 | 0.4 ± 0.1 |
| Muscle | 0.3 ± 0.1 | 0.4 ± 0.2 | 0.2 ± 0.3 | 0.3 ± 0.3 | 0.1 ± 0.2 | 0.1 ± 0.2 |
| Blood | 0.5 ± 0.2 | 0.8 ± 0.3 | 0.5 ± 1.1 | 0.8 ± 1.1 | 0.4 ± 0.8 | 0.5 ± 0.9 |
| Bone | 0.4 ± 0.3 | 0.6 ± 0.3 | 0.3 ± 0.4 | 0.5 ± 0.4 | 0.0 ± 0.0 | 0.1 ± 0.1 |
| Brain | 0.0 ± 0.0 | 0.1 ± 0.0 | 0.0 ± 0.0 | 0.1 ± 0.0 | 0.0 ± 0.0 | 0.0 ± 0.0 |
| Thyroid | 0.1 ± 0.2 | 1.1 ± 0.7 | 0.0 ± 0.3 | 1.5 ± 0.4 | 0.2 ± 0.3 | 2.7 ± 1.2 |
| Tumor | 11.9 ± 4.4 | 11.9 ± 4.0 | 10.9 ± 2.4 | 10.6 ± 2.1 | 4.8 ± 1.1 | 4.1 ± 1.0 |

^{a}% ID/g, mean ± SD, n = 5

TABLE 5
iso-[²¹¹At]AGMB-PODS-5F7GGC
(HER2-positive BT474 xenografts)
Percent injected dose per gramª-Single label study

| Tissue | 1 h | 4 h | 24 h^{b} |
|---|---|---|---|
| Liver | 8.4 ± 1.3 | 2.8 ± 0.8 | 0.6 ± 0.1 |
| Spleen | 1.4 ± 0.5 | 1.1 ± 0.4 | 0.3 ± 0.1 |
| Lungs | 2.6 ± 0.7 | 1.7 ± 0.4 | 0.4 ± 0.2 |
| Heart | 0.8 ± 0.2 | 0.4 ± 0.1 | 0.2 ± 0.1 |
| Kidneys | 70.9 ± 12.0 | 11.6 ± 2.4 | 2.5 ± 0.2 |
| Stomach | 3.2 ± 1.2 | 4.9 ± 1.5 | 1.0 ± 0.3 |
| Sm. Int. | 5.1 ± 0.5 | 2.7 ± 1.9 | 0.4 ± 0.1 |
| Lg. Int. | 0.6 ± 0.1 | 12.6 ± 4.5 | 1.0 ± 0.4 |
| Muscle | 0.5 ± 0.3 | 0.3 ± 0.3 | 0.0 ± 0.1 |
| Blood | 0.7 ± 0.3 | 0.3 ± 0.1 | 0.1 ± 0.1 |
| Bone | 0.7 ± 0.3 | 0.5 ± 0.4 | 0.1 ± 0.1 |
| Brain | 0.1 ± 0.1 | 0.1 ± 0.0 | 0.0 ± 0.0 |
| Thyroid | 0.8 ± 0.3 | 1.2 ± 0.4 | 1.1 ± 0.5 |
| Tumor | 15.1 ± 3.3 | 20.5 ± 2.4^{c} | 8.8 ± 0.8 |

^{a}% ID/g, mean ± SD, n = 5;
^{b}n = 4;
^{c}n = 2

A biodistribution study was performed to directly compare the in vivo performance of iso-[¹²⁵I]GMIB-PODS-5F7GGC and [¹⁷⁷Lu]Lu-DOTA-PODS-5F7GGC in athymic mice bearing BT474 xenografts (Table 6). The tumor uptake values for ¹²⁵I were 34±8% and 21±3% higher than those for ¹⁷⁷Lu at 1 and 4 h, respectively, but 26 8% lower at 24 h. While the radioactivity levels in the kidneys were comparable at 1 h (P>0.05), they were substantially lower for iso-[¹²⁵I]GMIB-PODS-5F7GGC (6.3±1.2% ID/g and 1.1±0.2% ID/g) than for [¹⁷⁷Lu]Lu-DOTA-PODS-5F7GGC (64.8±13.7% ID/g and 40.8±9.7% ID/g) at 4 and 24 h, respectively (P<0.0001 at both time points). As a result, the tumor-to-kidney activity concentration ratios were not significantly different between the two conjugates at 1 h (0.3±0.1 for ¹²⁵I and 0.2±0.1 for ¹⁷⁷Lu; P>0.05). However, at 4 and 24 h, the tumor-to-kidney activity concentration ratios for iso-[¹²⁵I]GMIB-PODS-5F7GGC were 2.9±0.7 and 5.8±2.0, considerably higher than those for [¹⁷⁷Lu]Lu-DOTA-PODS-5F7GGC—0.2±0.0 and 0.2±0.1 (P<0.0001 for both time points).

TABLE 6
iso-[¹²⁵I] GMIB-PODS-5F7GGC/[¹⁷⁷Lu]Lu-DOTA-PODS-5F7GGC
(HER2-positive BT474 xenografts)
Percent injected dose per gram^{a}

| | 1 h | | 4 h | | 24 h | |
|---|---|---|---|---|---|---|
| Tissue | ¹²⁵I | ¹⁷⁷Lu | ¹²⁵I | ¹⁷⁷Lu | ¹²⁵I | ¹⁷⁷Lu |
| Liver | 7.8 ± 2.6 | 1.3 ± 0.7 | 1.4 ± 0.4 | 0.6 ± 0.2 | 0.2 ± 0.1 | 0.4 ± 0.0 |
| Spleen | 0.6 ± 0.2 | 0.5 ± 0.2 | 0.1 ± 0.0 | 0.2 ± 0.1 | 0.0 ± 0.0 | 0.2 ± 0.1 |
| Lungs | 4.1 ± 2.6 | 1.7 ± 0.2 | 3.1 ± 1.3 | 1.6 ± 0.8 | 0.8 ± 0.9 | 0.4 ± 0.4 |
| Heart | 0.5 ± 0.2 | 0.6 ± 0.2 | 0.1 ± 0.0 | 0.3 ± 0.1 | 0.0 ± 0.0 | 0.2 ± 0.1 |
| Kidneys | 65.1 ± 20.0 | 73.6 ± 13.9 | 6.3 ± 1.2 | 64.8 ± 13.7 | 1.1 ± 0.2 | 40.8 ± 9.7 |
| Stomach | 1.4 ± 1.5 | 1.2 ± 1.7 | 0.4 ± 0.3 | 0.3 ± 0.2 | 0.0 ± 0.0 | 0.0 ± 0.0 |
| Sm. Int. | 5.1 ± 1.2 | 1.5 ± 1.3 | 2.9 ± 1.2 | 0.4 ± 0.2 | 0.1 ± 0.0 | 0.1 ± 0.0 |
| Lg. Int. | 0.5 ± 0.1 | 0.3 ± 0.1 | 11.1 ± 4.0 | 1.0 ± 0.2 | 0.3 ± 0.2 | 0.1 ± 0.0 |
| Muscle | 0.5 ± 0.1 | 0.5 ± 0.2 | 0.1 ± 0.0 | 0.1 ± 0.1 | 0.0 ± 0.0 | 0.1 ± 0.0 |
| Blood | 0.8 ± 0.2 | 1.6 ± 0.3 | 0.1 ± 0.1 | 0.6 ± 0.1 | 0.1 ± 0.1 | 0.3 ± 0.2 |
| Bone | 0.9 ± 0.5 | 0.9 ± 0.6 | 0.1 ± 0.1 | 0.2 ± 0.0 | 0.0 ± 0.0 | 0.0 ± 0.1 |
| Brain | 0.1 ± 0.0 | 0.1 ± 0.0 | 0.0 ± 0.0 | 0.0 ± 0.0 | 0.0 ± 0.0 | 0.0 ± 0.0 |
| Thyroid | 0.2 ± 0.2 | 0.2 ± 0.2 | 0.1 ± 0.0 | 0.1 ± 0.1 | 0.1 ± 0.0 | 0.1 ± 0.1 |
| Tumor | 21.2 ± 8.4 | 15.9 ± 6.2 | 17.4 ± 2.3 | 14.4 ± 1.9 | 6.4 ± 1.7 | 8.5 ± 1.5 |

^{a}% ID/g, mean ± SD, n = 5

### DISCUSSION

Recombinant methods were used to add a GGC sequence at the C-terminus of the anti-HER2 sdAb 5F7 to introduce a single cysteine for site-specific labeling. Our initial approach utilized an [¹³¹I]iodobenzoyl maleimido D-amino acid peptide-bearing prosthetic agent. Unfortunately, however, poor conjugation yields necessitated the modification of the sdAb with 2-iminothiolane so sufficient product for could be obtained for biological evaluation. As a result, the bioconjugation was no longer site specific. Maleimidoethyl 3-(guanidinomethyl)-5-[¹³¹I]iodobenzoate (MEGMIB) a maleimido analogue of the residualizing prosthetic agent iso-SGMIB was synthesized and evaluated for the site-specific labeling of 5F7GGC. Both 5F7 labeled randomly on its lysines using iso-[¹³¹I]SGMIB and [¹³¹]MEGMIB-5F7GGC exhibited excellent tumor targeting in vivo. However, the maleimido version had higher activity retention in the liver, spleen, and kidneys at 24 h post-injection, suggesting different metabolic patterns for the two radioimmunoconjugates. The extension of this labeling strategy to ²¹¹At was briefly evaluated via the synthesis of maleimidoethyl 3-[²¹¹At]astato-5-(guanidinomethyl)iodobenzoate ([²¹¹At]MEAGMB), but the in vivo results were inconclusive. These results can be explained by the rapid breakdown of [²¹¹At]MEAGMB-5F7GGC in the presence of endogenously abundant thiol-containing species.

A site-specific labeling strategy for sdAbs was developed with high in vivo stability applicable both to both the highly versatile array of iodine radionuclides and ²¹¹At for targeted alpha particle therapy. Although sdAbs labeled randomly on their lysines with reagents like iso-[¹³¹I]SGMIB can exhibit high affinity and stability, this has often involved selecting an sdAb without a lysine in its CDR regions or making an analogue in which a CDR lysine is removed. Radiohalogens produced as described herein may demonstrate features such as efficient and rapid conjugation at biologically relevant pH, and/or improved in vitro and in vivo stability compared with corresponding conjugates labeled using maleimide chemistry. In particular, a PODS moiety was combined with previously validated residualizing prosthetic agents iso-[¹³¹I]SGMIB (29) and iso-[²¹¹At]SAGMB (6) to derive iso-[¹³¹I]GMIB-PODS and iso-[²¹¹At]AGMB-PODS, respectively. 5F7-GGC labeled with these novel synthons were compared directly with conjugates synthesized using the maleimide-based prosthetic groups [¹³¹I]MEGMIB and [²¹¹At]MEAGMB. In addition, direct comparisons were also made between iso-[¹²⁵I]GMIB-PODS-5F7GGC and 5F7GGC labeled with ¹⁷⁷Lu using DOTA-PODS, i.e., a paired-label comparison of an sdAb labeled with a radiometal and radiohalogen.

The tin precursor Boc₂-iso-GMTB-PODS and the iodinated standard iso-GMIB-PODS were synthesized in reasonable yields in two steps. The syntheses of iso-[¹³¹I]GMIB-PODS, iso-[²¹¹At]AGMB-PODS, and iso-[²¹¹At]MEAGMB were performed in 2 steps in a manner similar to reported procedures for iso-[²¹¹At]SAGMB and [¹³¹I]MEGMIB. The conjugation of iso-GMIB-PODS to 5F7GGC gave higher yield than that of MEGMIB to 5F7GGC. Similarly, the conjugation of iso-[¹³¹I]GMIB-PODS to 5F7GGC had a higher RCY (P=0.0016) than that for [¹³¹I]MEGMIB (16). No significant difference in RCY was observed for the conjugation of iso-[¹³¹I]GMIB-PODS and iso-[²¹¹At]AGMB-PODS to 5F7GGC. The construction of [¹⁷⁷Lu]Lu-DOTA-PODS-5F7GGC was evaluated in two ways: (1) the synthesis and purification of DOTA-PODS-5F7GGC for direct labeling with [¹⁷⁷Lu]LuCl₃ and (2) the synthesis and purification of [¹⁷⁷Lu]Lu-DOTA-PODS for bioconjugation to 5F7GGC. It was observed that if DOTA-PODS-5F7GGC was labeled directly with [¹⁷⁷Lu]LuCl₃, some [¹⁷⁷Lu]Lu³⁺ became loosely bound to DOTA-PODS-5F7GGC, which disassociated when injected into animals. A possible explanation is that the [¹⁷⁷Lu]LuCl₃ formed colloids at neutral pH and co-eluted with the radioimmunoconjugate from the PD-10 column. [¹⁷⁷Lu]Lu-DOTA-PODS-5F7GGC was subsequently performed in two steps to avoid this problem. All non-radioactive versions of these sdAb immunoconjugates exhibited high binding affinity to the extracellular domains of HER2, demonstrating that conjugation with these prosthetic moieties did not diminish HER2 recognition. Moreover, the immunoreactivities and binding affinities to HER2-expressing cancer cells for all the radioimmunoconjugates were high and consistent with those reported previously for other 5F7 radioconjugates.

All of the radioimmunoconjugates in the study exhibited high uptake and internalization into HER2-positive BT474 cells, though some significant differences in behavior were observed. Compared with [¹²⁵I]MEGMIB-5F7GGC, iso-[¹³¹I]GMIB-PODS-5F7GGC showed higher intracellular retention after a 4 h incubation at 37° C., which likely reflected the higher in vitro stability of iso-[¹³¹I]GMIB-PODS-5F7GGC compared with [¹²⁵I]MEGMIB-5F7GGC. In a prior study, [¹³¹I]MEGMIB-5F7GGC was less stable in vitro than iso-[¹²⁵I]SGMIB-5F7, possibly due to hydrolysis and metabolism of the maleimido conjugate. Similar differences in behavior were seen when trastuzumab radioiodinated using D-amino acid residualizing peptide conjugates linked both via an active ester and a maleimide moiety were compared. The cellular uptake and intracellular residualization of iso-[²¹¹At]AGMB-PODS-5F7GGC was not significantly different than that for iso-[¹³¹I]GMIB-PODS-5F7GGC, consistent with a low degree of dehalogenation for both conjugates under in vitro conditions.

The contribution of the PODS moiety to the stability of the radioimmunoconjugates under in vitro conditions was investigated by comparing the behavior of iso-[²¹¹At]AGMB-PODS-5F7GGC and iso-[²¹¹At]MEAGMB-5F7GGC in three media, two of which contained thiol-bearing substances that are conducive to retro-Michael-mediated degradative processes. Excellent stability was observed for iso-[²¹¹At]AGMB-PODS-5F7GGC in PBS, cysteine, and HSA. In contrast, iso-[²¹¹At]MEAGMB-5F7GGC quickly degraded within 3 h in both cysteine and HSA. Interestingly, however, significantly higher stability was seen earlier for iso-[¹³¹I]MEGMIB-5F7GGC, with 90% of the radioimmunoconjugate intact after a 24-h incubation in human serum (16). The considerably lower stability of iso-[²¹¹At]MEAGMB-5F7GGC compared to iso-[¹³¹I]MEGMIB-5F7GGC could relate to several factors, such as a higher dehalogenation of the [²¹¹At]MEAGMB moiety either before or after disassociation from the sdAb conjugate.

Because of its poor in vitro stability, iso-[²¹¹At]MEAGMB-5F7GGC was not evaluated further in biodistribution studies. Except for a confirmatory single-label evaluation of iso-[²¹¹At]AGMB-PODS-5F7GGC, biodistribution experiments were done in paired label format. This facilitated the direct comparison of two radioimmunoconjugates while eliminating potentially confounding variables (such as tumor size) that can exist between groups of experimental animals. When iso-[¹³¹I]GMIB-PODS-5F7GGC and iso-[¹²⁵I]MEGMIB-5F7GGC were compared in tumor-bearing mice, there was a trend towards higher tumor uptake for the PODS conjugate at 1 and 4 h, but the differences were not significant. On the other hand, the maleimide-based conjugate exhibited more than two-fold lower kidney activity levels at the 1 h time point in both tumor-bearing and non-tumor bearing mice but not thereafter; by 24 h, the opposite behavior was observed. The low initial kidney uptake of iso-[¹²⁵I]MEGMIB-5F7GGC is consistent with results published previously, which suggest that this behavior may reflect the in vivo lability of the thiosuccinimide linkage generating rapidly excreted labeled catabolites.

Studies in mice with HER2-expressing xenografts with both randomly labeled 5F7 and the closely related VHH_1028 sdAb have demonstrated considerably greater therapeutic effectiveness for the ²¹¹At-labeled compared with the ¹³¹I-labeled conjugates. (VHH_1028 sdAb is described in the publication to Feng et al. “Evaluation of an ¹³¹I-labeled HER2-specific single domain antibody fragment for the radiopharmaceutical therapy of HER2-expressing cancers” Sci Rep 12, 3020 (2022), which is incorporated herein by reference. As such, site-specific and biologically stable reagents for labeling sdAbs with ²¹¹At were developed. In order to evaluate the effect of halogen on in vivo behavior, iso-[²¹¹At]AGMB-PODS-5F7GGC and iso-[¹³¹I]GMIB-PODS-5F7GGC were compared in paired-label format. No significant differences in tumor uptake between ²¹¹At and ¹³¹I were observed, although ²¹¹At levels were significantly higher than those for ¹³¹I in spleen, stomach, and thyroid, results that are consistent with a higher degree of dehalogenation for the ²¹¹At-labeled sdAb. Similar halogen-dependent trends in tumor and normal tissue uptake were reported for 5F7 randomly labeled using iso-[²¹¹At]SAGMB and iso-[¹³¹I]SGMIB in SCID mice with BT474 xenografts (Choi et al. “Astatine-211 labeled anti-HER2 5F7 single domain antibody fragment conjugates: radiolabeling and preliminary evaluation” Nucl. Med. Biol. 2018; 56:10-20). On the other hand, the biodistribution of iso-[²¹¹At]AGMB-VHH_1028 an sdAb differing from 5F7 by only one amino acid and exhibiting identical in vivo behavior has been determined in single-label format in athymic mice with BT474 subcutaneous xenografts. When iso-[²¹¹At]AGMB-PODS-5F7GGC was evaluated in single-label format, its tumor uptake was about twice than that reported for iso-[²¹¹At]AGMB-VHH_1028 in the same model. Moreover, levels of ²¹¹At in the stomach, thyroid, and spleen were about two times lower for the PODS-based radioimmunoconjugate suggesting it was more stable in vivo than the iso-[²¹¹At]SAGMB conjugate.

Finally, the use of PODS for site-specific labeling produces only a single species, providing an opportunity to compare metal and halogen sdAb labeling strategies while excluding heterogeneity as a confounding variable. This is particularly important for sdAbs that bind internalizing targets like HER2 because a labeled sdAb bearing a metal-free chelate could have altered charge and potentially different internalization and/or trapping in tumor cells. Significantly higher tumor uptake was observed for iso-[¹²⁵I]GMIB-PODS-5F7GGC at 1 and 4 h compared with [¹⁷⁷Lu]Lu-DOTA-PODS-5F7GGC, with the opposite behavior seen at 24 h. One possible limitation of the radioiodinated sdAb is that it showed higher hepatobiliary organ uptake at the earlier time points. Kidney retention of ¹⁷⁷Lu was substantially higher than for the radioiodinated agent, with the difference reaching a factor of about 40 at 24 h. The potentially dose-limiting renal activity levels are in line with those reported previously for other sdAbs labeled with ¹⁷⁷Lu. The results of this head-to-head comparison suggest that for 5F7 (and perhaps other sdAbs), radiohalogenation using our PODS-bearing residualizing agent might be preferable to the use of a radiometal.

Additional information is available in US Provisional Application Nos. 62/507,477 (filed May 17, 2017) and 62/634,385 (filed Feb. 23, 2018), and U.S. Pat. No. 11,000,604 (filed May 17, 2018, issued May 11, 2021 and entitled “Reagent for Site-Selective Bioconjugates of Proteins or Antibodies”), the contents of which are hereby incorporated by reference.

This written description uses examples to disclose the present disclosure, including the best mode, and also to enable any person skilled in the art to practice the invention, including making and using any devices or systems and performing any incorporated methods. The patentable scope of the present disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A compound having the structure (I): 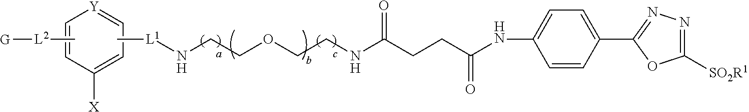
where:
a is 1-6; b is 1-6; c is 1-6;
L¹ is —C(O)— or 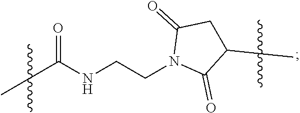 [SMILES: *C(=O)NCCN1C(=O)CC(*)C1=O]
L² is a bond or —(CH₂)ₙ—, where n is 1-6;
G is guanidine, a guanidine having one or more carbamate protecting groups, PO₃H, SO₃H, PO₂—OtBu, SO₂—OtBu, arginine, phosphono-phenylalanine, sulfo-phenylalanine, glutamate, aspartate, lysine, a hydrophilic carbohydrate moiety, or a polyethylene glycol (PEG) chain;
Y is CH or N;
X is SnR²₃, B(OH)₂, Bpin, or a radiohalogen;
R¹ is C₁₋₆ alkyl; and
each R² is independently C₁₋₆ alkyl.

2. The compound of claim 1, wherein the compound has the structure (II): 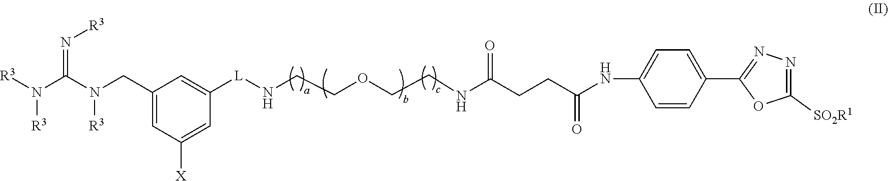
where:
a is 1-6; b is 1-6; c is 1-6;
L is —C(O)— or 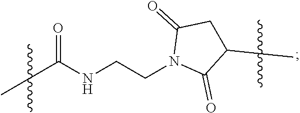 [SMILES: *C(=O)NCCN1C(=O)CC(*)C1=O]
X is SnR²₃, B(OH)₂, Bpin, or a radiohalogen;
R¹ is C₁₋₆ alkyl;
each R² is independently C₁₋₆ alkyl; and
each R³ is independently a carbamate protecting group or H.

3. The compound of claim 2, where the compound has the structure (III):

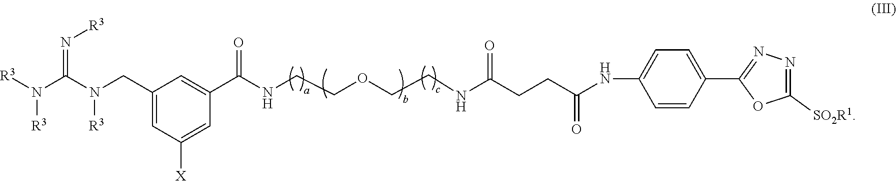

4. The compound of claim 2, where the compound has the structure (IV).

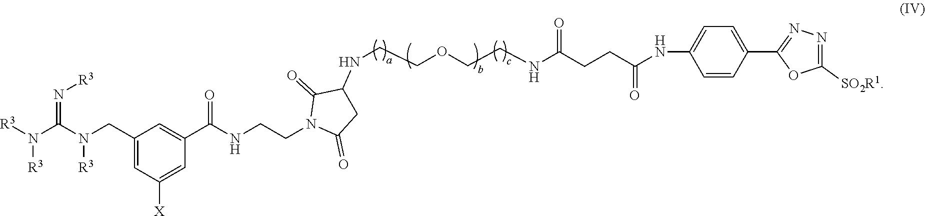

5. The compound of any one of claims 1-4, wherein R¹ is methyl, ethyl, or propyl.

6. The compound of any one of claims 1-5, wherein a is 1 or 2; b is 2 or 3; and c is 1 or 2.

7. The compound of any one of claims 1-6, where X is SnR²₃, B(OH)₂, or Bpin; R² is methyl, ethyl, or n-butyl.

8. The compound of any one of claims 1-7, where X is a radiohalogen, and the radiohalogen is selected from the group consisting of ¹⁸F, ¹²²I, ¹²³I, ¹²⁴I, ¹²⁵I, ¹³¹I, ⁷⁵Br, ⁷⁶Br, ⁷⁷Br, ^{80m}Br, and ²¹¹At.

9. A radiolabeled protein or peptide comprising one or more radiohalogens coupled to a protein or peptide through a thioether bond, wherein the one or more radiohalogenated prosthetic agent conjugates have structure (V), or the one or more radiohalogenated prosthetic agent conjugates are a pharmaceutically acceptable salt of structure (V): 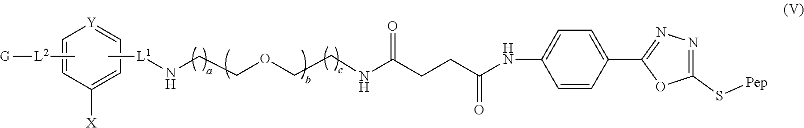
where:
a is 1-6; b is 1-6; c is 1-6;
L¹ is —C(O)— or 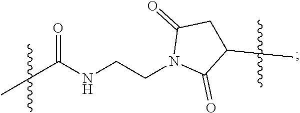 [SMILES: *C(=O)NCCN1C(=O)CC(*)C1=O]
L² is a bond or —(CH₂)ₙ—, where n is 1-6;
G is guanidine, a guanidine having one or more carbamate protecting groups, PO₃H, SO₃H, PO₂—OtBu, SO₂—OtBu, arginine, phosphono-phenylalanine, sulfo-phenylalanine, glutamate, aspartate, lysine, a hydrophilic carbohydrate moiety, or a polyethylene glycol (PEG) chain;
Y is CH or N;
X is a radiohalogen;
R¹ is C₁₋₆ alkyl; and
Pep is a protein or a peptide.

10. The radiolabeled protein/peptide of claim 9, wherein the radiolabeled protein/peptide has the structure (VI): 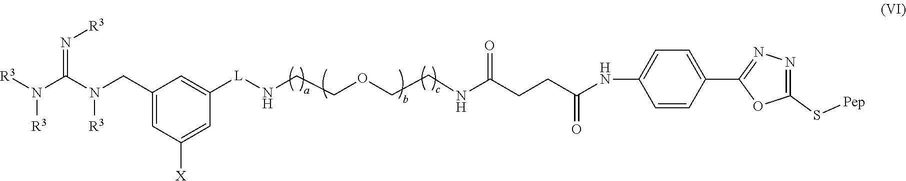
where:
a is 1-6; b is 1-6; c is 1-6;
L is —C(O)— or 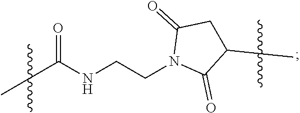 [SMILES: *C(=O)NCCN1C(=O)CC(*)C1=O]
X is a radiohalogen;
R¹ is C₁₋₆ alkyl; and
each R³ is independently a carbamate protecting group or H.

11. The radiolabeled protein/peptide of claim 10, wherein the radiolabeled protein/peptide has the structure (VII): 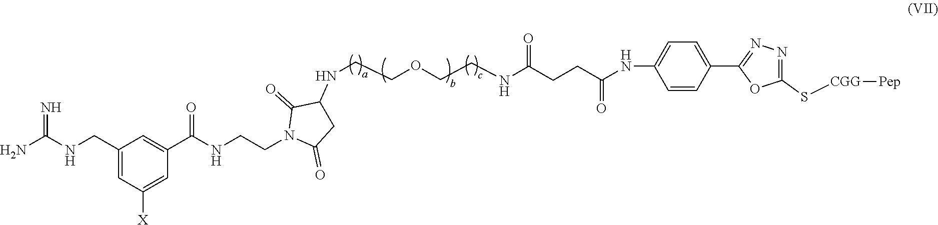
where Pep is a protein/peptide.

12. The radiolabeled protein/peptide of claim 10, wherein the radiolabeled protein/peptide has the structure (VIII): 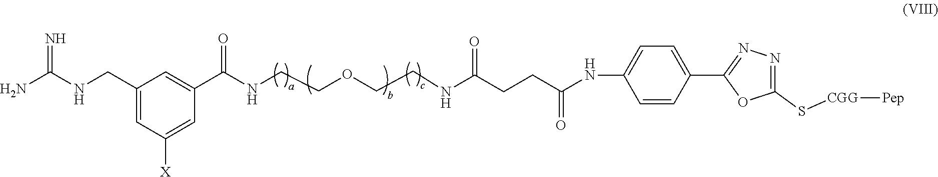
where Pep is a protein/peptide.

13. The radiolabeled protein/peptide of any one of claims 9-12, wherein the radiohalogen is selected from the group consisting of ¹⁸F, ¹²²I, ¹²³I, ¹²⁴I, ¹²⁵I, ¹³¹I, ⁷⁵Br, ⁷⁶Br, ⁷⁷Br, ^{80m}Br, or ²¹¹At.

14. The radiolabeled protein/peptide of any one of claims 9-13, wherein the protein/peptide comprises at least one cysteine residue and wherein one or more of the radiolabels are coupled to the protein/peptide through the cysteine residue.

15. The radiolabeled protein/peptide of claim 14, wherein the protein/peptide comprises a C-terminal GGC tail and wherein the radiolabel is coupled to the protein/peptide through the C-terminal GGC tail.

16. The radiolabeled protein/peptide of any one of claims 9-15, wherein the protein/peptide is an antibody.

17. The radiolabeled protein/peptide of any one of claims 9-15, wherein the protein/peptide is a single domain antibody fragment.

18. The radiolabeled protein/peptide of any one of claims 9-17, wherein the protein/peptide is a tumor targeting protein/peptide.

19. A method of performing molecular imaging in a subject, comprising administering to the subject an effective amount of a radiolabeled protein/peptide as described in any one of claims 8-18.

20. The method of claim 19, wherein molecular imaging is performed to detect cancer cells.

21. The method of claim 20, wherein the cancer cell are breast cancer cells.

22. A method of treating cancer in a subject, comprising administering to the subject an effective amount of a radiolabeled protein/peptide as described in any one of claims 8-18.

23. The method of claim 22, wherein the cancer is breast cancer.

24. A method of labeling a protein or a peptide with a radiohalogen, the method comprising:
(i) obtaining a compound having the structure (II): 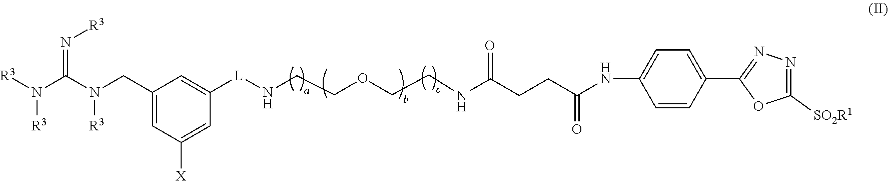
where:
a is 1-6; b is 1-6; c is 1-6;
X is a radiohalogen;
R¹ is C₁₋₆ alkyl;
each R³ is independently a carbamate protecting group or H; and
L is C(O) or 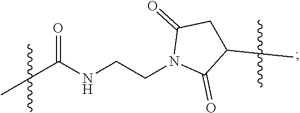 [SMILES: *C(=O)NCCN1C(=O)CC(*)C1=O]
and
(ii) when R³ is a H, reacting the compound (II) with the protein or the peptide, or when R³ is a carbamate protecting group, reacting compound (II) with the peptide.

25. The method of claim 24, further comprising removing carbamate protecting groups from the compound prior to reacting the compound with the protein/peptide.

26. The method of claim 25, wherein the protein/peptide is an antibody.

27. The method of claim 25, wherein the protein/peptide is a single domain antibody fragment.

28. The method of any one of claims 24-27, wherein the protein/peptide is a tumor targeting protein/peptide.

29. The method of any one of claims 24-28, further comprising modifying a protein/peptide by adding a C-terminal GGC tail to the protein/peptide, wherein the compound reacts with the cysteine residue of the GGC tail to form a thioether bond between the compound and the protein/peptide.

30. The method of claim 24, wherein the radiolabeled protein/peptide has the structure (VII): 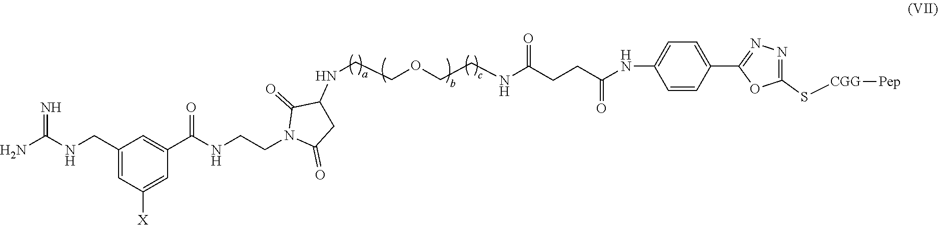
where Pep is the protein/peptide.

31. The method of claim 24, wherein the radiolabeled protein/peptide has the structure (VIII): 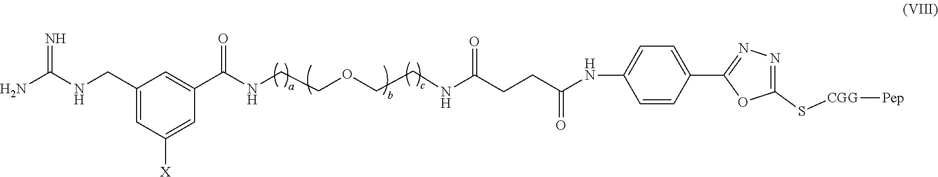
where Pep is the protein/peptide.
